SEQUENCE LISTING (56 sequences)

SEQ ID NO: 1 - DNA, 60 bp, Homo sapiens
atggagacagacacactgctcctgtgggtcctgctcctgtgggtccccggaagcacaggc

SEQ ID NO: 2 - DNA, 135 bp, Homo sapiens
accacaacccctgcccctagacctcccacacccgctcccacaatcgctagccagcctctgtccctgaggcccgaggcctgcagacctgccgctggcggagccgtccacacaagaggactggacttcgcttgcgac

SEQ ID NO: 3 - DNA, 63 bp, Homo sapiens
ttctggctccccattggctgcgctgccttcgtcgtggtctgcattctgggatgcattctgatt

SEQ ID NO: 4 - DNA, 114 bp, Homo sapiens
tgctggctgacaaagaaaaagtactccagctccgtgcacgaccctaacggagagtacatgttcatgagggccgtcaacacagccaagaaaagcaggctgacagacgtcaccctc

SEQ ID NO: 5 - DNA, 336 bp, Homo sapiens
agagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 6 - DNA, 513 bp, Artificial Sequence
  Combination of sequences
ttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 7 - DNA, 63 bp, Artificial Sequence
  Self-cleaving sequence
ggctccggcgagggcaggggaagtcttctaacatgcggggacgtggaggaaaatcccggccca

SEQ ID NO: 8 - DNA, 66 bp, Artificial Sequence
  Self-cleaving sequence
ggcagcggcgccaccaacttttccctgctgaagcaggccggcgacgtggaagagaaccccgggcct

SEQ ID NO: 9 - DNA, 69 bp, Artificial Sequence
  Self-cleaving sequence
ggatctggacagtgtaccaattacgctctgctcaagctcgccggagacgtcgagtccaaccctggacct

SEQ ID NO: 10 - DNA, 75 bp, Artificial Sequence
  Self-cleaving sequence
ggatctggagtgaaacagacactgaatttcgatctgctcaagctcgccggagacgtcgagtccaaccctggacct

SEQ ID NO: 11 - DNA, 75 bp, Homo sapiens
atgtttcacgtcagctttagatatatctttggcctcccccctctgattctggtcctgctccccgtcgcctcctcc

SEQ ID NO: 12 - DNA, 456 bp, Homo sapiens
gactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcat

SEQ ID NO: 13 - DNA, 270 bp, Homo sapiens
accacaacccctgcccccaggcctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgat

SEQ ID NO: 14 - DNA, 72 bp, Homo sapiens
atctacatttgggctcccctcgccggaacctgcggagtgctcctgctcagcctcgtgattaccctctactgc

SEQ ID NO: 15 - protein, 20 aa, Homo sapiens
METDTLLLWVLLLWVPGSTG

SEQ ID NO: 16 - protein, 45 aa, Homo sapiens
TTTPAPRPPTPAPTIASQPLSLRPEACRPAAGGAVHTRGLDFACD

SEQ ID NO: 17 - protein, 21 aa, Homo sapiens
FWLPIGCAAFVVVCILGCILI

SEQ ID NO: 18 - protein, 38 aa, Homo sapiens
CWLTKKKYSSSVHDPNGEYMFMRAVNTAKKSRLTDVTL

SEQ ID NO: 19 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 171 aa, Artificial Sequence
  Combination of sequences
FWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 21 aa, Artificial Sequence
  Self-cleaving sequence
GSGEGRGSLLTCGDVEENPGP

SEQ ID NO: 22 - protein, 22 aa, Artificial Sequence
  Self-cleaving sequence
GSGATNFSLLKQAGDVEENPGP

SEQ ID NO: 23 - protein, 23 aa, Artificial Sequence
  Self-cleaving sequence
GSGQCTNYALLKLAGDVESNPGP

SEQ ID NO: 24 - protein, 25 aa, Artificial Sequence
  Self-cleaving sequence
GSGVKQTLNFDLLKLAGDVESNPGP

SEQ ID NO: 25 - protein, 25 aa, Homo sapiens
MFHVSFRYIFGLPPLILVLLPVASS

SEQ ID NO: 26 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 27 - protein, 90 aa, Homo sapiens
TTTPAPRPPTPAPTIASQPLSLRPEACRPAAGGAVHTRGLDFACDTTTPAPRPPTPAPTIASQPLSLRPEACRPAAGGAVHTRGLDFACD

SEQ ID NO: 28 - protein, 24 aa, Homo sapiens
IYIWAPLAGTCGVLLLSLVITLYC

SEQ ID NO: 29 - DNA, 1443 bp, Artificial Sequence
  Full humanized anti-CD19 single CAR
atggagaccgataccctcctgctctgggtcctgctcctgtgggtccccggaagcacaggcgacattcagatgacacagtccccctccagcctcagcgctagcgtcggcgacagggtgacaatcacatgcagggcctcccaggacattagcaagtacctcaactggtatcagcagaagcctggcaaggctcccaagctcctcatctaccacacaagcaggctgcactccggcgtcccctccagattcagcggctccggctccggcacagactacacactgacaatctccagcctccagcctgaggacttcgctacctactactgccagcagggaaacacactgccctacacattcggaggcggaaccaaggtcgagattaagggaagcacctccggcggcggcagcggaggcggaagcggcggaggaggctccagccaggtccagctccaggagtccggccctggcctcgtgaagcctagccagacactgtccctgacatgcacagtgtccggcgtcagcctccccgactacggagtgtcctggattagacagcctcccggaaagggactggagtggctcggcgtcatctggggaagcgagacaacctactacaactccgccctcaagtccagactcaccattagcagggacaactccaagaacacactgtacctccagatgaactccctgagggccgaggacacagccgtctactactgcgctaagcactactactacggaggctcctacgctatggactactggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 30 - protein, 481 aa, Artificial Sequence
  Full humanized anti-CD19 single CAR
METDTLLLWVLLLWVPGSTGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKGSTSGGGSGGGSGGGGSSQVQLQESGPGLVKPSQTLSLTCTVSGVSLPDYGVSWIRQPPGKGLEWLGVIWGSETTYYNSALKSRLTISRDNSKNTLYLQMNSLRAEDTAVYYCAKHYYYGGSYAMDYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 2382 bp, Artificial Sequence
  Full humanized anti-CD19/mb-IL-7 dual signal CAR
atggagaccgataccctcctgctctgggtcctgctcctgtgggtccccggaagcacaggcgacattcagatgacacagtccccctccagcctcagcgctagcgtcggcgacagggtgacaatcacatgcagggcctcccaggacattagcaagtacctcaactggtatcagcagaagcctggcaaggctcccaagctcctcatctaccacacaagcaggctgcactccggcgtcccctccagattcagcggctccggctccggcacagactacacactgacaatctccagcctccagcctgaggacttcgctacctactactgccagcagggaaacacactgccctacacattcggaggcggaaccaaggtcgagattaagggaagcacctccggcggcggcagcggaggcggaagcggcggaggaggctccagccaggtccagctccaggagtccggccctggcctcgtgaagcctagccagacactgtccctgacatgcacagtgtccggcgtcagcctccccgactacggagtgtcctggattagacagcctcccggaaagggactggagtggctcggcgtcatctggggaagcgagacaacctactacaactccgccctcaagtccagactcaccattagcagggacaactccaagaacacactgtacctccagatgaactccctgagggccgaggacacagccgtctactactgcgctaagcactactactacggaggctcctacgctatggactactggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatgtttcacgtcagctttagatatatctttggcctcccccctctgattctggtcctgctccccgtcgcctcctccgactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcataccacaacccctgcccccaggcctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgctga

SEQ ID NO: 32 - protein, 793 aa, Artificial Sequence
  Full humanized anti-CD19/mb-IL-7 dual signal CAR
METDTLLLWVLLLWVPGSTGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKGSTSGGGSGGGSGGGGSSQVQLQESGPGLVKPSQTLSLTCTVSGVSLPDYGVSWIRQPPGKGLEWLGVIWGSETTYYNSALKSRLTISRDNSKNTLYLQMNSLRAEDTAVYYCAKHYYYGGSYAMDYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHTTTPAPRPPTPAPTIASQPLSLRPEACRPAAGGAVHTRGLDFACDTTTPAPRPPTPAPTIASQPLSLRPEACRPAAGGAVHTRGLDFACDIYIWAPLAGTCGVLLLSLVITLYC

SEQ ID NO: 33 - DNA, 63 bp, Homo sapiens
atggctctgcctgtgacagccctgctcctgcccctcgccctgctcctccacgctgccaggccc

SEQ ID NO: 34 - protein, 21 aa, Homo sapiens
MALPVTALLLPLALLLHAARP

SEQ ID NO: 35 - DNA, 2514 bp, Artificial Sequence
  Full humanized anti-CD19/anti-CD20 dual signal and dual CAR
atggagaccgataccctcctgctctgggtcctgctcctgtgggtccccggaagcacaggcgacattcagatgacacagtccccctccagcctcagcgctagcgtcggcgacagggtgacaatcacatgcagggcctcccaggacattagcaagtacctcaactggtatcagcagaagcctggcaaggctcccaagctcctcatctaccacacaagcaggctgcactccggcgtcccctccagattcagcggctccggctccggcacagactacacactgacaatctccagcctccagcctgaggacttcgctacctactactgccagcagggaaacacactgccctacacattcggaggcggaaccaaggtcgagattaagggaagcacctccggcggcggcagcggaggcggaagcggcggaggaggctccagccaggtccagctccaggagtccggccctggcctcgtgaagcctagccagacactgtccctgacatgcacagtgtccggcgtcagcctccccgactacggagtgtcctggattagacagcctcccggaaagggactggagtggctcggcgtcatctggggaagcgagacaacctactacaactccgccctcaagtccagactcaccattagcagggacaactccaagaacacactgtacctccagatgaactccctgagggccgaggacacagccgtctactactgcgctaagcactactactacggaggctcctacgctatggactactggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatggctctgcctgtgacagccctgctcctgcccctcgccctgctcctccacgctgccaggcccgagattgtgctcacccagtcccccgctaccctcagcctcagccctggcgagagggccacaatgacatgcagggcctccagctccgtgaactacatggactggtatcagcagaagcctggccaagctcccagaccttggatctacgctacctccaacctcgcctccggcgtccccgctagattcagcggctccggctccggcacagacttcacactgacaatctccagcctggagcctgaggacttcgctgtgtactactgccagcagtggtccttcaaccctcccacattcggaggcggaaccaaggtcgagattaagggctccacaagcggaggaggctccggcggaggctccggaggcggcggaagctcccaggtccagctcgtgcagtccggcgctgaggtcaagaagcctggcgctagcgtcaaggtcagctgcaaggctagcggatacaccttcacctcctacaacatgcactgggtcagacaagcccctggacagggcctggagtggattggcgctatctaccctggcaacggagacacaagctacaaccagaagttcaagggaaaggctaccctcaccagggacacaagcacaagcacagtgtacatggagctcagctccctgaggagcgaggacacagccgtctactactgcgctagatccaactactacggaagctcctactggttcttcgacgtctggggacagggaaccacagtgacagtgtccagcacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 36 - protein, 838 aa, Artificial Sequence
  Full humanized anti-CD19/anti-CD20 dual signal and dual CAR
METDTLLLWVLLLWVPGSTGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKGSTSGGGSGGGSGGGGSSQVQLQESGPGLVKPSQTLSLTCTVSGVSLPDYGVSWIRQPPGKGLEWLGVIWGSETTYYNSALKSRLTISRDNSKNTLYLQMNSLRAEDTAVYYCAKHYYYGGSYAMDYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEIVLTQSPATLSLSPGERATMTCRASSSVNYMDWYQQKPGQAPRPWIYATSNLASGVPARFSGSGSGTDFTLTISSLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWIGAIYPGNGDTSYNQKFKGKATLTRDTSTSTVYMELSSLRSEDTAVYYCARSNYYGSSYWFFDVWGQGTTVTVSSTTTPAPRPPTPAPTIASQPLSLRPEACRPAAGGAVHTRGLDFACDIYIWAPLAGTCGVLLLSLVITLYC

SEQ ID NO: 37 - DNA, 1446 bp, Artificial Sequence
  Full humanized anti-CD20 single CAR
atggagacagacacactccttctctgggtcctgctcctgtgggtccccggaagcacaggcgagattgtgctcacccagtcccccgctaccctcagcctcagccctggcgagagggccacaatgacatgcagggcctccagctccgtgaactacatggactggtatcagcagaagcctggccaagcccctagaccttggatctacgctacctccaacctcgcctccggcgtccccgctagattcagcggctccggctccggcacagacttcacactgacaatctccagcctggagcctgaggacttcgctgtgtactactgccagcagtggtccttcaaccctcccacattcggaggcggaaccaaggtcgagattaagggaagcacctccggcggcggcagcggaggaggctccggaggcggaggctccagccaggtccagctcgtgcagtccggcgctgaggtcaagaagcctggcgctagcgtcaaggtcagctgcaaggctagcggatacaccttcacctcctacaacatgcactgggtcagacaagcccctggacagggcctggagtggattggcgctatctaccctggcaacggagacacaagctacaaccagaagttcaagggaaaggctaccctcaccagggacacaagcacaagcacagtgtacatggagctcagctccctgaggagcgaggacacagccgtctactactgcgctagatccaactactacggaagctcctactggttcttcgacgtctggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 38 - protein, 482 aa, Artificial Sequence
  Full humanized anti-CD20 single CAR
METDTLLLWVLLLWVPGSTGEIVLTQSPATLSLSPGERATMTCRASSSVNYMDWYQQKPGQAPRPWIYATSNLASGVPARFSGSGSGTDFTLTISSLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWIGAIYPGNGDTSYNQKFKGKATLTRDTSTSTVYMELSSLRSEDTAVYYCARSNYYGSSYWFFDVWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1449 bp, Artificial Sequence
  Full humanized anti-CD22 L-H single CAR
atggagaccgataccctcctgctctgggtcctgctcctgtgggtccccggaagcacaggcgaggacattcagatgacacagtccccctccagcctcagcgctagcgtcggcgatagagtcaccatgtcctgcaaaagctcccagtccgtgctctactccgccaatcacaaaaactatctggcttggtatcagcaaaagcctggcaaagcccctaagctcctgatctactgggctagcacaagagaaagcggagtgcctagcaggttcagcggcagcggctccggcacagactttacctttaccattagctccctgcaacccgaagacattgccacatactattgccatcagtatctgagcagctggacattcggaggcggaaccaaggtcgagattaagggaagcacctccggcggcggcagcggaggaggctccggaggcggaggctccagccaggtccagctcgtgcaaagcggagccgaagtgaaaaagcctggcgctagcgtcaaggtcagctgtaaggctagcggatacacattcacaagctattggctccactgggtcagacaagcccctggccaaggcctcgagtggattggctatatcaatcccaggaacgattacacagaatacaatcagaatttcaaagacaaagccacactgacagccgataagtccacctccaccgcttacatggaactgtccagcctcagatccgaggataccgctgtgtattactgtgccaggagagacatcacaaccttctattggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 40 - protein, 483 aa, Artificial Sequence
  Full humanized anti-CD22 L-H single CAR
METDTLLLWVLLLWVPGSTGEDIQMTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVEIKGSTSGGGSGGGSGGGGSSQVQLVQSGAEVKKPGASVKVSCKASGYTFTSYWLHWVRQAPGQGLEWIGYINPRNDYTEYNQNFKDKATLTADKSTSTAYMELSSLRSEDTAVYYCARRDITTFYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 1455 bp, Artificial Sequence
  Full humanized anti-CD22 H-L single CAR
atggagaccgataccctcctgctctgggtcctgctcctgtgggtccccggaagcacaggcgaggtccagctcgtggaaagcggaggcggagtggtcagacctggcggaagcctcagactcagctgtgctgcctccggcgatagcgtcagctccaactccgccgcttggaattggattagacaagcccctggcaaaggcctcgagtggctcggcaggacctattacaggagcaaatggtataacgattacgctgtgtccgtgaaaagcaggatcacaatctcccccgatacctccaagaatagcttttacctccagatgaatagcctcaagacagaggataccgctgtgtattactgtgccagggaggtcaccggagacctcgaggatgcctttgacatttggggacagggaaccacagtgacagtgtccagcggaagcacctccggcggcggcagcggaggaggctccggaggcggaggctccagcgacattcagatgacacagtccccctccagcctcagcgctagcgtcggcgatagagtcaccattacctgtagagctagccaaaccatttggtcctacctcaactggtatcagcaaaagcctggcaaagcccctaagctcctgatctacgctgccagcagcctccagtccggcgtcccctccaggttctccggcagcggcagcggaaccgatttcacactgacaatctccagcctccagcctgaggatttcgctacctattactgtcagcaaagctatagcattccccaaacctttggccaaggcacaaagctcgagattaagaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 42 - protein, 485 aa, Artificial Sequence
  Full humanized anti-CD22 H-L single CAR
METDTLLLWVLLLWVPGSTGEVQLVESGGGVVRPGGSLRLSCAASGDSVSSNSAAWNWIRQAPGKGLEWLGRTYYRSKWYNDYAVSVKSRITISPDTSKNSFYLQMNSLKTEDTAVYYCAREVTGDLEDAFDIWGQGTTVTVSSGSTSGGGSGGGSGGGGSSDIQMTQSPSSLSASVGDRVTITCRASQTIWSYLNWYQQKPGKAPKLLIYAASSLQSGVPSRFSGSGSGTDFTLTISSLQPEDFATYYCQQSYSIPQTFGQGTKLEIKTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 2382 bp, Artificial Sequence
  Full humanized anti-CD20/mb-IL-7 dual signal CAR
atggagacagacacactccttctctgggtcctgctcctgtgggtccccggaagcacaggcgagattgtgctcacccagtcccccgctaccctcagcctcagccctggcgagagggccacaatgacatgcagggcctccagctccgtgaactacatggactggtatcagcagaagcctggccaagcccctagaccttggatctacgctacctccaacctcgcctccggcgtccccgctagattcagcggctccggctccggcacagacttcacactgacaatctccagcctggagcctgaggacttcgctgtgtactactgccagcagtggtccttcaaccctcccacattcggaggcggaaccaaggtcgagattaagggaagcacctccggcggcggcagcggaggaggctccggaggcggaggctccagccaggtccagctcgtgcagtccggcgctgaggtcaagaagcctggcgctagcgtcaaggtcagctgcaaggctagcggatacaccttcacctcctacaacatgcactgggtcagacaagcccctggacagggcctggagtggattggcgctatctaccctggcaacggagacacaagctacaaccagaagttcaagggaaaggctaccctcaccagggacacaagcacaagcacagtgtacatggagctcagctccctgaggagcgaggacacagccgtctactactgcgctagatccaactactacggaagctcctactggttcttcgacgtctggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatgtttcacgtcagctttagatatatctttggcctcccccctctgattctggtcctgctccccgtcgcctcctccgactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcataccacaacccctgcccctagacctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 44 - protein, 794 aa, Artificial Sequence
  Full humanized anti-CD20/mb-IL-7 dual signal CAR
METDTLLLWVLLLWVPGSTGEIVLTQSPATLSLSPGERATMTCRASSSVNYMDWYQQKPGQAPRPWIYATSNLASGVPARFSGSGSGTDFTLTISSLEPEDFAVYYCQQWSFNPPTFGGGTKVEIKGSTSGGGSGGGSGGGGSSQVQLVQSGAEVKKPGASVKVSCKASGYTFTSYNMHWVRQAPGQGLEWIGAIYPGNGDTSYNQKFKGKATLTRDTSTSTVYMELSSLRSEDTAVYYCARSNYYGSSYWFFDVWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHTTTPAPRPPTPAPTIASQPLSLRPEACRPAAGGAVHTRGLDFACDTTTPAPRPPTPAPTIASQPLSLRPEACRPAAGGAVHTRGLDFACDIYIWAPLAGTCGVLLLSLVITLYC

SEQ ID NO: 45 - DNA, 2385 bp, Artificial Sequence
  Full anti-CD22 L-H/mb-IL-7 dual signal CAR
atggagaccgataccctcctgctctgggtcctgctcctgtgggtccccggaagcacaggcgaggacattcagatgacacagtccccctccagcctcagcgctagcgtcggcgatagagtcaccatgtcctgcaaaagctcccagtccgtgctctactccgccaatcacaaaaactatctggcttggtatcagcaaaagcctggcaaagcccctaagctcctgatctactgggctagcacaagagaaagcggagtgcctagcaggttcagcggcagcggctccggcacagactttacctttaccattagctccctgcaacccgaagacattgccacatactattgccatcagtatctgagcagctggacattcggaggcggaaccaaggtcgagattaagggaagcacctccggcggcggcagcggaggaggctccggaggcggaggctccagccaggtccagctcgtgcaaagcggagccgaagtgaaaaagcctggcgctagcgtcaaggtcagctgtaaggctagcggatacacattcacaagctattggctccactgggtcagacaagcccctggccaaggcctcgagtggattggctatatcaatcccaggaacgattacacagaatacaatcagaatttcaaagacaaagccacactgacagccgataagtccacctccaccgcttacatggaactgtccagcctcagatccgaggataccgctgtgtattactgtgccaggagagacatcacaaccttctattggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatgtttcacgtcagctttagatatatctttggcctcccccctctgattctggtcctgctccccgtcgcctcctccgactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcataccacaacccctgcccctagacctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 46 - protein, 795 aa, Artificial Sequence
  Full anti-CD22 L-H/mb-IL-7 dual signal CAR
METDTLLLWVLLLWVPGSTGEDIQMTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVEIKGSTSGGGSGGGSGGGGSSQVQLVQSGAEVKKPGASVKVSCKASGYTFTSYWLHWVRQAPGQGLEWIGYINPRNDYTEYNQNFKDKATLTADKSTSTAYMELSSLRSEDTAVYYCARRDITTFYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHTTTPAPRPPTPAPTIASQPLSLRPEACRPAAGGAVHTRGLDFACDTTTPAPRPPTPAPTIASQPLSLRPEACRPAAGGAVHTRGLDFACDIYIWAPLAGTCGVLLLSLVITLYC

SEQ ID NO: 47 - DNA, 2391 bp, Artificial Sequence
  Full anti-CD22 H-L/mb-IL-7 dual signal CAR
atggagaccgataccctcctgctctgggtcctgctcctgtgggtccccggaagcacaggcgaggtccagctcgtggaaagcggaggcggagtggtcagacctggcggaagcctcagactcagctgtgctgcctccggcgatagcgtcagctccaactccgccgcttggaattggattagacaagcccctggcaaaggcctcgagtggctcggcaggacctattacaggagcaaatggtataacgattacgctgtgtccgtgaaaagcaggatcacaatctcccccgatacctccaagaatagcttttacctccagatgaatagcctcaagacagaggataccgctgtgtattactgtgccagggaggtcaccggagacctcgaggatgcctttgacatttggggacagggaaccacagtgacagtgtccagcggaagcacctccggcggcggcagcggaggaggctccggaggcggaggctccagcgacattcagatgacacagtccccctccagcctcagcgctagcgtcggcgatagagtcaccattacctgtagagctagccaaaccatttggtcctacctcaactggtatcagcaaaagcctggcaaagcccctaagctcctgatctacgctgccagcagcctccagtccggcgtcccctccaggttctccggcagcggcagcggaaccgatttcacactgacaatctccagcctccagcctgaggatttcgctacctattactgtcagcaaagctatagcattccccaaacctttggccaaggcacaaagctcgagattaagaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatgtttcacgtcagctttagatatatctttggcctcccccctctgattctggtcctgctccccgtcgcctcctccgactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcataccacaacccctgcccctagacctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 48 - protein, 797 aa, Artificial Sequence
  Full anti-CD22 H-L/mb-IL-7 dual signal CAR
METDTLLLWVLLLWVPGSTGEVQLVESGGGVVRPGGSLRLSCAASGDSVSSNSAAWNWIRQAPGKGLEWLGRTYYRSKWYNDYAVSVKSRITISPDTSKNSFYLQMNSLKTEDTAVYYCAREVTGDLEDAFDIWGQGTTVTVSSGSTSGGGSGGGSGGGGSSDIQMTQSPSSLSASVGDRVTITCRASQTIWSYLNWYQQKPGKAPKLLIYAASSLQSGVPSRFSGSGSGTDFTLTISSLQPEDFATYYCQQSYSIPQTFGQGTKLEIKTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHTTTPAPRPPTPAPTIASQPLSLRPEACRPAAGGAVHTRGLDFACDTTTPAPRPPTPAPTIASQPLSLRPEACRPAAGGAVHTRGLDFACDIYIWAPLAGTCGVLLLSLVITLYC

SEQ ID NO: 49 - DNA, 2517 bp, Artificial Sequence
  Full anti-CD19/anti-CD22 L-H dual signal and dual CAR
atggagaccgataccctcctgctctgggtcctgctcctgtgggtccccggaagcacaggcgacattcagatgacacagtccccctccagcctcagcgctagcgtcggcgacagggtgacaatcacatgcagggcctcccaggacattagcaagtacctcaactggtatcagcagaagcctggcaaggctcccaagctcctcatctaccacacaagcaggctgcactccggcgtcccctccagattcagcggctccggctccggcacagactacacactgacaatctccagcctccagcctgaggacttcgctacctactactgccagcagggaaacacactgccctacacattcggaggcggaaccaaggtcgagattaagggaagcacctccggcggcggcagcggaggcggaagcggcggaggaggctccagccaggtccagctccaggagtccggccctggcctcgtgaagcctagccagacactgtccctgacatgcacagtgtccggcgtcagcctccccgactacggagtgtcctggattagacagcctcccggaaagggactggagtggctcggcgtcatctggggaagcgagacaacctactacaactccgccctcaagtccagactcaccattagcagggacaactccaagaacacactgtacctccagatgaactccctgagggccgaggacacagccgtctactactgcgctaagcactactactacggaggctcctacgctatggactactggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggcagcggcgccaccaacttttccctgctgaagcaggccggcgacgtggaagagaaccccgggcctatggctctgcctgtgacagccctgctcctgcccctcgccctgctcctccacgctgccaggcccgacattcagatgacacagtccccctccagcctcagcgctagcgtcggcgatagagtcaccatgtcctgcaaaagctcccagtccgtgctctactccgccaatcacaaaaactatctggcttggtatcagcaaaagcctggcaaagcccctaagctcctgatctactgggctagcacaagagaaagcggagtgcctagcaggttcagcggcagcggctccggcacagactttacctttaccattagctccctgcaacccgaagacattgccacatactattgccatcagtatctgagcagctggacattcggaggcggaaccaaggtcgagattaagggctccacaagcggaggaggctccggcggaggctccggaggcggcggaagctcccaggtccagctcgtgcaaagcggagccgaagtgaaaaagcctggcgctagcgtcaaggtcagctgtaaggctagcggatacacattcacaagctattggctccactgggtcagacaagcccctggccaaggcctcgagtggattggctatatcaatcccaggaacgattacacagaatacaatcagaatttcaaagacaaagccacactgacagccgataagtccacctccaccgcttacatggaactgtccagcctcagatccgaggataccgctgtgtattactgtgccaggagagacatcacaaccttctattggggacagggaaccacagtgacagtgtccagcacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 50 - protein, 839 aa, Artificial Sequence
  Full anti-CD19/anti-CD22 L-H dual signal and dual CAR
METDTLLLWVLLLWVPGSTGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKGSTSGGGSGGGSGGGGSSQVQLQESGPGLVKPSQTLSLTCTVSGVSLPDYGVSWIRQPPGKGLEWLGVIWGSETTYYNSALKSRLTISRDNSKNTLYLQMNSLRAEDTAVYYCAKHYYYGGSYAMDYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVEIKGSTSGGGSGGGSGGGGSSQVQLVQSGAEVKKPGASVKVSCKASGYTFTSYWLHWVRQAPGQGLEWIGYINPRNDYTEYNQNFKDKATLTADKSTSTAYMELSSLRSEDTAVYYCARRDITTFYWGQGTTVTVSSTTTPAPRPPTPAPTIASQPLSLRPEACRPAAGGAVHTRGLDFACDIYIWAPLAGTCGVLLLSLVITLYC

SEQ ID NO: 51 - DNA, 2526 bp, Artificial Sequence
  Full anti-CD19/anti-CD22 H-L dual signal and dual CAR
atggagaccgataccctcctgctctgggtcctgctcctgtgggtccccggaagcacaggcgacattcagatgacacagtccccctccagcctcagcgctagcgtcggcgacagggtgacaatcacatgcagggcctcccaggacattagcaagtacctcaactggtatcagcagaagcctggcaaggctcccaagctcctcatctaccacacaagcaggctgcactccggcgtcccctccagattcagcggctccggctccggcacagactacacactgacaatctccagcctccagcctgaggacttcgctacctactactgccagcagggaaacacactgccctacacattcggaggcggaaccaaggtcgagattaagggaagcacctccggcggcggcagcggaggcggaagcggcggaggaggctccagccaggtccagctccaggagtccggccctggcctcgtgaagcctagccagacactgtccctgacatgcacagtgtccggcgtcagcctccccgactacggagtgtcctggattagacagcctcccggaaagggactggagtggctcggcgtcatctggggaagcgagacaacctactacaactccgccctcaagtccagactcaccattagcagggacaactccaagaacacactgtacctccagatgaactccctgagggccgaggacacagccgtctactactgcgctaagcactactactacggaggctcctacgctatggactactggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggcagcggcgccaccaacttttccctgctgaagcaggccggcgacgtggaagagaaccccgggcctatggctctgcctgtgacagccctgctcctgcccctcgccctgctcctccacgctgccaggcccgaggtccagctcgtggaaagcggaggcggagtggtcagacctggcggaagcctcagactcagctgtgctgcctccggcgatagcgtcagctccaactccgccgcttggaattggattagacaagcccctggcaaaggcctcgagtggctcggcaggacctattacaggagcaaatggtataacgattacgctgtgtccgtgaaaagcaggatcacaatctcccccgatacctccaagaatagcttttacctccagatgaatagcctcaagacagaggataccgctgtgtattactgtgccagggaggtcaccggagacctcgaggatgcctttgacatttggggacagggaaccacagtgacagtgtccagcggaagcacctccggcggcggcagcggaggaggctccggaggcggaggctccagcgacattcagatgacacagtccccctccagcctcagcgctagcgtcggcgatagagtcaccattacctgtagagctagccaaaccatttggtcctacctcaactggtatcagcaaaagcctggcaaagcccctaagctcctgatctacgctgccagcagcctccagtccggcgtcccctccaggttctccggcagcggcagcggaaccgatttcacactgacaatctccagcctccagcctgaggatttcgctacctattactgtcagcaaagctatagcattccccaaacctttggccaaggcacaaagctcgagattaagacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 52 - protein, 842 aa, Artificial Sequence
  Full anti-CD19/anti-CD22 H-L dual signal and dual CAR
METDTLLLWVLLLWVPGSTGDIQMTQSPSSLSASVGDRVTITCRASQDISKYLNWYQQKPGKAPKLLIYHTSRLHSGVPSRFSGSGSGTDYTLTISSLQPEDFATYYCQQGNTLPYTFGGGTKVEIKGSTSGGGSGGGSGGGGSSQVQLQESGPGLVKPSQTLSLTCTVSGVSLPDYGVSWIRQPPGKGLEWLGVIWGSETTYYNSALKSRLTISRDNSKNTLYLQMNSLRAEDTAVYYCAKHYYYGGSYAMDYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLVESGGGVVRPGGSLRLSCAASGDSVSSNSAAWNWIRQAPGKGLEWLGRTYYRSKWYNDYAVSVKSRITISPDTSKNSFYLQMNSLKTEDTAVYYCAREVTGDLEDAFDIWGQGTTVTVSSGSTSGGGSGGGSGGGGSSDIQMTQSPSSLSASVGDRVTITCRASQTIWSYLNWYQQKPGKAPKLLIYAASSLQSGVPSRFSGSGSGTDFTLTISSLQPEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYC

SEQ ID NO: 53 - DNA, 1449 bp, Artificial Sequence
  Full humanized anti-CD30 single CAR
atggagaccgataccctcctgctctgggtcctgctcctgtgggtccccggaagcacaggcgaggagattgtgctcacccaaagccctgccacactgtccctgtcccccggagagagggccacaatcaattgcaaagcctcccagtccgtggatttcgatggcgacagctatatgaattggtatcagcaaaagcctggcaaagcccctaagctgctcatctacgctgcctccaacctcgagtccggcattcccagcaggttcagcggaagcggcagcggaaccgatttcacactgacaatctccagcctccagcctgaggatttcgctacctattactgtcagcaaagcaatgaagacccctggaccttcggaggcggaaccaaggtcgagattaagggaagcacctccggcggcggcagcggaggaggctccggaggcggaggctccagccagattcagctcgtgcaaagcggagccgaagtgaaaaagcctggcgctagcgtcaaggtcagctgtaaggctagcggatacacattcacagactattacattacctgggtcagacaagcccctggccaaagactcgagtggattggctggatctaccctggctccggcaataccaaatacaatgagaaattcaaaggcaaagccacaatcacagtggatacctccgcctccaccgcttacatggaactgtccagcctcaggagcgaagacacagccgtctactattgcgctaactatggcaattactggtttgcctattggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccagg

SEQ ID NO: 54 - protein, 483 aa, Artificial Sequence
  Full humanized anti-CD30 single CAR
METDTLLLWVLLLWVPGSTGEEIVLTQSPATLSLSPGERATINCKASQSVDFDGDSYMNWYQQKPGKAPKLLIYAASNLESGIPSRFSGSGSGTDFTLTISSLQPEDFATYYCQQSNEDPWTFGGGTKVEIKGSTSGGGSGGGSGGGGSSQIQLVQSGAEVKKPGASVKVSCKASGYTFTDYYITWVRQAPGQRLEWIGWIYPGSGNTKYNEKFKGKATITVDTSASTAYMELSSLRSEDTAVYYCANYGNYWFAYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 2385 bp, Artificial Sequence
  Full anti-CD30/mb-IL-7 dual signal CAR
atggagaccgataccctcctgctctgggtcctgctcctgtgggtccccggaagcacaggcgaggagattgtgctcacccaaagccctgccacactgtccctgtcccccggagagagggccacaatcaattgcaaagcctcccagtccgtggatttcgatggcgacagctatatgaattggtatcagcaaaagcctggcaaagcccctaagctgctcatctacgctgcctccaacctcgagtccggcattcccagcaggttcagcggaagcggcagcggaaccgatttcacactgacaatctccagcctccagcctgaggatttcgctacctattactgtcagcaaagcaatgaagacccctggaccttcggaggcggaaccaaggtcgagattaagggaagcacctccggcggcggcagcggaggaggctccggaggcggaggctccagccagattcagctcgtgcaaagcggagccgaagtgaaaaagcctggcgctagcgtcaaggtcagctgtaaggctagcggatacacattcacagactattacattacctgggtcagacaagcccctggccaaagactcgagtggattggctggatctaccctggctccggcaataccaaatacaatgagaaattcaaaggcaaagccacaatcacagtggatacctccgcctccaccgcttacatggaactgtccagcctcaggagcgaagacacagccgtctactattgcgctaactatggcaattactggtttgcctattggggacagggaaccacagtgacagtgtccagcaccacaacccctgcccctagacctcccacacccgctcccacaatcgctagccagcctctgtccctgaggcccgaggcctgcagacctgccgctggcggagccgtccacacaagaggactggacttcgcttgcgacttctggctccccattggctgcgctgccttcgtcgtggtctgcattctgggatgcattctgatttgctggctgacaaagaaaaagtactccagctccgtgcacgaccctaacggagagtacatgttcatgagggccgtcaacacagccaagaaaagcaggctgacagacgtcaccctcagagtcaagttctccagatccgccgacgctcccgcttacaagcagggacagaaccagctctacaacgagctcaacctcggcaggagagaggaatacgacgtcctggacaagaggagaggaagagaccctgagatgggaggcaagcccagaaggaagaaccctcaggagggactgtacaacgagctccagaaggacaaaatggctgaggcttactccgagattggcatgaagggagagaggagaaggggcaagggacacgacggactgtaccagggactgtccaccgctaccaaggacacatacgacgctctgcacatgcaggctctgcctcccaggggctccggcgagggcaggggaagtcttctaacatgcggggacgtggaggaaaatcccggcccaatgtttcacgtcagctttagatatatctttggcctcccccctctgattctggtcctgctccccgtcgcctcctccgactgtgacattgagggaaaggatggcaaacagtatgagtccgtgctcatggtcagcattgaccaactgctcgactccatgaaagagattggctccaactgtctgaacaatgagttcaatttctttaagaggcacatttgcgatgccaataaggaaggcatgtttctgtttagagctgccaggaagctcagacaattcctcaaaatgaatagcacaggcgatttcgatctgcatctgctcaaggtcagcgaaggcacaaccattctgctcaactgtaccggacaggtcaagggaagaaaacccgctgccctcggcgaagcccaacccacaaagtccctggaagagaataagtccctgaaagagcaaaagaaactgaatgacctctgctttctgaaaagactcctgcaagagattaagacatgctggaataagattctgatgggaaccaaagagcataccacaacccctgcccctagacctcccacacccgctcccacaatcgctagccagcctctgtccctgaggcccgaggcctgcagacctgccgctggcggagccgtccacacaagaggactggacttcgcttgcgacacaaccacacccgctcccaggccccctacccctgcccctaccattgcctcccaacccctcagcctcagacctgaagcctgtaggcccgctgccggaggcgctgtgcataccaggggcctcgattttgcctgtgatatctacatttgggctcccctcgccggaacctgcggagtgctcctgctcagcctcgtgattaccctctactgc

SEQ ID NO: 56 - protein, 795 aa, Artificial Sequence
  Full anti-CD30/mb-IL-7 dual signal CAR
METDTLLLWVLLLWVPGSTGEEIVLTQSPATLSLSPGERATINCKASQSVDFDGDSYMNWYQQKPGKAPKLLIYAASNLESGIPSRFSGSGSGTDFTLTISSLQPEDFATYYCQQSNEDPWTFGGGTKVEIKGSTSGGGSGGGSGGGGSSQIQLVQSGAEVKKPGASVKVSCKASGYTFTDYYITWVRQAPGQRLEWIGWIYPGSGNTKYNEKFKGKATITVDTSASTAYMELSSLRSEDTAVYYCANYGNYWFAYWGQGTTVTVSSTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHTTTPAPRPPTPAPTIASQPLSLRPEACRPAAGGAVHTRGLDFACDTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2020/0038443 A1
(43) Pub. Date: Feb. 6, 2020

(54) MULTI-FUNCTION AND MULTI-TARGETING CAR SYSTEM AND METHODS FOR USE THEREOF

(71) Applicant: AbCyte Therapeutics Inc., San Jose, CA (US)

(72) Inventors: Yongke Zhang, Palo Alto, CA (US); Huijun Zhi, Clarksburg, MD (US)

(21) Appl. No.: 16/530,981

(22) Filed: Aug. 2, 2019

Related U.S. Application Data
(60) Provisional application No. 62/714,687, filed on Aug. 4, 2018.

(51) Int. Cl. A61K 35/17; C12N 15/85; C07K 14/725; C07K 14/715; C07K 14/47; C07K 14/54

(52) U.S. Cl. CPC A61K 35/17; C12N 15/85; C07K 14/7051; C07K 2319/03; C07K 14/4748; C07K 14/5418; C07K 2319/02; C07K 14/715

(57) ABSTRACT

The present invention provides compositions and methods for treating cancer in a human. The invention includes and relates to administering a genetically modified T cell to express a CAR system wherein the CAR system comprises a polynucleotide encoding multiple signaling-modules with either multiple antibody targeting tumor specific antigen or with membrane bound cytokine to further enhance immune cell survival and proliferation. The multi-costimulatory signaling structure gave less toxicity than conventional CAR-T structures using CD28 signaling domain. A CAR system with multiple antibodies targeting different tumor specific antigen can target difference cancer cell populations simultaneously.

### CROSS REFERENCE TO RELATED APPLICATION

This application claims priority to U.S. Provisional Application No. 62/714,687, filed Aug. 4, 2018, the disclosure of which is incorporated herein by reference in its entirety.

### BACKGROUND

T cells, a type of lymphocyte, play a central role in cell-mediated immunity. They are distinguished from other lymphocytes, such as B cells and natural killer cells (NK cells), by the presence of a T-cell receptor (TCR) on the cell surface. Once activated, these cells proliferate rapidly and secrete cytokines that regulate immune response. Memory T cells, a subset of T cells, persist long-term and respond to their cognate antigen, thus providing the immune system with “memory” against past infections and/or tumor cells.

T cells can be genetically engineered to produce special receptors on their surface called chimeric antigen receptors (CARs). CARs that redirect T-cell specificity to desired tumor-associated antigens (TAAs) (Eshhar Z, et al. 1993) are engineered to activate T cells for survival, serial killing, and cytokine production only upon contacting TAA (Savoldo B, et al. 2011). Adoptive transfer of CAR T cells can achieve durable complete responses in some patients; successful outcomes are associated with engraftment and long-term persistence of CAR T cells (Porter D L, et al. 2015). Long-term immunosurveillance by persisting CAR T cells is likely key to achieving durable responses in adoptive cell therapy (ACT). Memory T-cell subsets appear to exist along a gradient of differentiation characterized by reciprocal potentials for longevity and effector function. Indeed, adoptively transferred effector CD8+ T cells derived from central memory (TCM) or naive (TN) T-cell subsets in murine and nonhuman primate models demonstrated increased therapeutic potential. Thus, T-cell subsets corresponding to an immature state of differentiation are appealing for their potential to provide superior clinical utility (Berger C, et al. 2008; Hinrichs C S, et al. 2011.)

T-memory stem cells (TSCM), so far the least differentiated memory T-cell subset identified, can be generated under specific ex vivo culture conditions (e.g., IL-7, IL-15, or small molecules targeting metabolic or developmental pathways) (Cieri N, et al. 2013; Gattinoni L, et al. 2011; Sabatino M, et al. 2016). This memory subset possesses the highest self-renewal capacity and therapeutic potential. Due to superior persistence in the absence of antigen-driven stimulation, TSCM are suggested to be the primary precursors of T-cell memory once antigen is cleared in an immune response (Lugli E, et al. 2013). Furthermore, only the frequency of CD8+CD45RA+CCR7+ TSCM-like cells in the infusion product correlated with the expansion of CD19-specific CART cells (Xu Y, et al. 2014). Because TSCM represents only a small percentage (2-3%) of peripheral blood mononuclear cells (PBMCs), strategies to manufacture TSCM suitable for human applications are essential and under development.

Endogenous and administered T cells receive prosurvival signals through the common cytokine receptor γ-chain, such as those signals mediated by IL-2 and IL-7, independent of native or introduced immune-receptors. The common gamma chain (γc) (or CD132), also known as interleukin-2 receptor subunit gamma or IL-2RG, is a cytokine receptor sub-unit that is common to the receptor complexes for at least six different interleukin receptors: IL-2, IL-4, IL-7, IL-9, IL-15 and IL-21 receptor. The γc glycoprotein is a member of the type I cytokine receptor family expressed on most lymphocyte (white blood cell) populations, and its gene is found on the X-chromosome of mammals. This protein is located on the surface of immature blood-forming cells in bone marrow. One end of the protein resides outside the cell where it binds to cytokines and the other end of the protein resides in the interior of the cell where it transmits signals to the cell's nucleus. The common gamma chain partners with other proteins to direct blood-forming cells to form lymphocytes (a type of white blood cell). The receptor also directs the growth and maturation of lymphocyte subtypes: T cells, B cells, and natural killer cells. These cells kill viruses, make antibodies, and help regulate the entire immune system.

Interleukin-7 (IL-7) is a type I glycoprotein that is predicted to form a four-alpha-helix structure with a hydrophobic core. It is produced primarily by stromal cells and exerts its effects through a receptor complex consisting of IL-7 R alpha and common gamma-chain/IL-2 R gamma. IL-7 signaling is essential for the establishment and maintenance of normal immune system functions. It is required for mouse and human T cell development and homeostatic proliferation, mouse B cell development, and the generation of CD4+ and CD8+ memory T cells. IL-7 R alpha-deficient mice have reduced numbers of thymocytes, impaired T cell and B cell development, and lack gamma delta T cells, a small subset of T cells found in epithelium-rich tissues. The requirement of IL-7 for T cell survival has been partially attributed to its ability to induce expression of the anti-apoptotic Bcl-2, Bcl-xL, and Mcl-1 proteins. In addition, IL-7 plays a role in regulating V(D)J recombination at the TCR gamma, TCR beta, and immunoglobulin heavy chain loci. IL-15 is a prosurvival cytokine that is required for homeostatic maintenance of long-lived CD8+ memory T cells (Zhang X et al. 1998), inhibits activation-induced cell death (AICD) (Marks-Konczalik J, et al. 2000), enhances in vivo antitumor activity (Klebanoff C A, et al. 2004), and reverses T-cell energy (Teague R M, et al 2006). High IL15 expression in the tumor microenvironment correlates with elevated infiltration of CD3+ T cells, correlating with improved survival of patients with colorectal cancer. Moreover, IL-15 is required for the generation of innate-like T cells that participate in immunosurveillance and impede tumor growth (Dadi S, et al. 2016).

Clinical trials to date have shown chimeric antigen receptor (CAR) T cells to have great promise in hematologic malignancies resistant to standard chemotherapies. Most notably, CD19-specific CAR (CD19-CAR) T-cell therapies have had remarkable results including long-term remissions in B-cell malignancies (Kochenderfer J N, et al. 2010, Kalos M, et al. 2011, Porter D L, et al. 2011, Grupp S A, et al. 2013, Kochenderfer J N, et al. 2013, Maude S L, et al. 2014).

To date, current efforts have focused on CAR T-cells demonstrating efficacy in various B-cell malignancies. While initial remission rates of approximately 90% are common in B-ALL using CD19-CAR, most of these relapse within a year. The relapse is at least in part due to the antigen escape. Thus, targeting single antigen carries the risk of immune escape and this could be overcome by targeting multiple desired antigens, especially in solid tumor with higher tumor heterogeneity. Therefore, there remains a need for improved chimeric antigen receptor-based therapies that allow for more effective, safe, and efficient targeting of various cancers such as B-cell associated malignancies (ALL, CLL and NHL), multiple myeloma, AML, lymphoma as well as many other solid tumors.

### SUMMARY OF INVENTION

In one aspect of the invention, a multi-function/multi-targeting module structure of a CAR system comprises a plural of genes encoding two or more CARs targeting multiple tumor-specific antigens to target different population of cancer cells simultaneously. In another aspect of the invention, a multi-function/mono-targeting module structure of a CAR system comprises one gene encoding a CAR targeting a tumor-specific antigen and another gene encoding a co-stimulatory molecule comprising membrane-bound cytokine/cytokine receptor to enhance cancer-targeting immune cell survival and proliferation further. In another aspect of the invention, a method to improve the persistence and potential for the memory of an engineered T cells and harness interleukin autocrine loop signaling comprising engineering T cells with co-expression of a recombinant membrane-bound variant of IL-7, IL-15, IL-12, IL-2, IL22 or IL17 linked to a second-generation CAR intracellular signaling domain using the a lentiviral system.

In another aspect of the invention, an isolated polynucleotide of a multi-function/signaling-module CAR system comprises a first gene encoding a first polypeptide and a second gene encoding a second polypeptide, wherein said first polypeptide comprising five or more following: (i) a signal peptide, (ii) a binding protein, (iii) a hinge region, (iv) a transmembrane domain, (v) a co-stimulatory domain of ICOS and (vi) a CD3 zeta signaling domain; and said second polypeptide comprising five or more following: (i) a signal peptide, (ii) a binding protein, (iii) a hinge region, (iv) a transmembrane domain, wherein at least one of the binding protein binds to an antigen on cancer cells. In another aspect of the invention, both first and second polypeptides are co-stimulatory molecules. In further another aspect of the invention, said first and said second polypeptide are co-expressed at same or a similar level by linking the first gene and second gene with a 2A peptide gene. The hinge regions are optional in some embodiments.

In another aspect of the invention, the binding protein of said first polypeptide of a multi-function/signaling-module CAR system can be an antigen recognition domain while the binding protein of said second polypeptide can be an immuno-regulatory cytokine or cytokine receptor. In another aspect of the invention, that both first and second polypeptides are co-stimulatory molecules.

In another aspect of the invention, the binding protein of said first polypeptide a multi-function/signaling-module CAR system can be an immuno-regulatory cytokine or cytokine receptor and the binding protein of said second polypeptide can be an antigen recognition domain, wherein said second polypeptide is a CAR. In further another aspect of the invention, both first and second polypeptides are co-stimulatory molecules.

In another aspect of the invention, the binding protein of both said first polypeptide and said second polypeptide can be an antigen recognition domain. In further another aspect, both first and second co-stimulatory molecule can be a CAR.

In another aspect of the invention, an immuno-regulatory cytokines or an extracellular domain of cytokine receptors can be linked to a transmembrane domain and a co-stimulatory domain to form a co-stimulatory molecule.

In yet another aspect of the invention, a lentivirus can be used for expression of a multi-function CAR system.

In yet another aspect of the invention, said vector further comprises a polynucleotide comprising an inducible suicide gene.

In yet another aspect of the invention, in a dual-co-stimulatory molecule CAR-T cell, one co-stimulatory molecule contains antigen recognition domain targeting against target selected from a group consisting of Methothelin, Muc 16, Claudin 18.2, Claudin 8, NY-ESO-1, CD 19, CD 20, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2, Her2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGSS), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAXS, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR), while the other co-stimulatory molecule can contain another antigen recognition domain or contain an immuno-regulatory cytokine/a cytokine receptor selected from a group consisting of membrane bound IL-7, IL-21, IL-15, IL-12, IL-2, IL-17 TGFb receptor and IL15Ra sushi domain. In yet another aspect of the invention, the dual-co-stimulatory molecule CAR-T cell can be used for treating lymphoma, leukemia and various solid tumors originated from lung, breast, prostate, colon, kidney, ovary, head and neck, liver, pancreas, bile duct and brain.

In yet another aspect of the invention, the engineered T cells comprises a CAR gene and another gene encoding the membrane-bound IL-7 without the costimulatory signal 2; wherein the CAR gene and membrane-bound IL-7 gene are linked by a 2A peptide gene.

In another aspect of the invention, the T cells can be engineered to have both endogenous autocrine loop-signaling and co-stimulatory signaling from membrane-bound IL-7 (mbIL-7) by transduction of a lentivirus vector comprising a polynucleotide encoding a co-stimulatory molecule comprising an IL-7 or an extracellular domain of IL-7 receptor.

In another aspect of the invention, a method for stimulating a T cell-mediated immune response to a target cell population or tissue in a human, the method comprising administering to the human an effective amount of an engineered cell genetically modified to express a first CAR and a second CAR wherein the first CAR comprises a CD19 antigen binding domain, a transmembrane domain, a costimulatory signaling region comprising ICOS, and a CD3 Zeta signaling domain, and wherein the second CAR comprises an immuno-regulatory cytokines or extracellular domain of cytokine receptors and a transmembrane domain.

In another aspect of the invention, a method for stimulating a T cell-mediated immune response to a target cell population or tissue in a human, the method comprising administering to the human an effective amount of an engineered cell genetically modified to express a first polypeptide and a second polypeptide wherein the first polypeptide comprises a CD19 antigen binding domain, and a transmembrane domain, and wherein the second polypeptide comprises an immuno-regulatory cytokines or extracellular domain of cytokine receptors and a transmembrane domain. The immuno-regulatory cytokines can be IL-7. The CD19 antigen binding domain can specifically bind to cancer cells expressing CD19.

In another aspect of the invention, a vector comprising the polynucleotide of this invention is also provided.

In another aspect of the invention, an engineered cell comprising the expression vector of this invention is also provided. In some embodiments, the first antigen recognition domain binds to CD19; and the second antigen recognition domain binds to CD20 or CD22. In some embodiments, first antigen recognition domain binds to BCMA; and the second antigen recognition domain binds to CD38, CD138, or CS 1. In some embodiments, the first antigen recognition domain binds to CD123; and the second antigen recognition domain binds to CD33 or CLL1. In some embodiments, the first antigen recognition domain binds to PSCA; and the second antigen recognition domain binds to PSMA.

In another aspect of the invention, an engineered cell comprising the vector of this invention is a T-cell or NK cell. In some embodiments, the engineered cell comprises inactivated gene of PD-1, TIM3, or LAGS by gene knockout. In some embodiments, the engineered NK cell is an NKT cell or NK-92 cell.

In another aspect of the invention, a polynucleotide comprising sequence encoding the co-stimulatory molecules of the invention, the polynucleotide comprises a sequence encoding an antigen recognition domain of a scFv or a VHH nanobody.

In another aspect of the invention, a pharmaceutical composition comprising the cell of the invention is also provided.

In another aspect of the invention, a method for treating cancer, the method comprises administering to a subject in need thereof, a therapeutically effective amount of the cell of the invention. In some embodiments, the cancer is blood cancer. In some embodiments, the cancer is lymphoma.

In another aspect of the invention, a polypeptide of a single CAR or a CAR system, the polypeptide comprises a sequence selected from a group consisting of SEQ ID NO:20, 30, 32, 36, 38, 40, 42, 44, 46, 48, 50, 52, 54, and 56.

In another aspect of the invention, a polynucleotide encoding a single CAR or CAR system, the polynucleotide comprises a sequence selected from a group consisting of SEQ ID NO:6, 29, 31, 35, 37, 39, 41, 43, 45, 47, 49, 51, 53 and 55.

In another aspect of the invention, an expression vector comprises the polynucleotide sequence of 6, 29, 31, 35, 37, 39, 41, 43, 45, 47, 49, 51, 53 or 55.

In another aspect of the invention, an engineered cell comprises the expression vector.

In another aspect of the invention, a composition comprises the engineered cell.

In another aspect of the invention, a pharmaceutical composition comprises the engineered cell and a pharmaceutically acceptable carrier.

In yet another aspect of the invention, a method for treating B cell lymphomas (NHL), acute lymphoblastic leukemia (ALL) and chronic lymphocytic leukemia (CLL) comprising administering to a subject in need thereof, a therapeutically effective amount of the engineered cell expressing the polypeptide of SEQ ID NO: 30 or SEQ ID NO:32.

In yet another aspect of the invention, a method for treating B cell lymphomas (NHL) and chronic lymphocytic leukemia (CLL) comprising administering to a subject in need thereof, a therapeutically effective amount of the engineered cell expressing the polypeptide of SEQ ID NO:38 or SEQ ID NO:44.

In yet another aspect of the invention, a method for treating B cell lymphomas (NHL), acute lymphoblastic leukemia (ALL) and chronic lymphocytic leukemia (CLL) comprising administering to a subject in need thereof, a therapeutically effective amount of the engineered cell expressing a polypeptide with a sequence selected from a group consisting of SEQ ID NO:40, SEQ ID NO:42, SEQ ID NO:46, and SEQ ID NO:48.

In yet another aspect of the invention, a method for treating Hodgkin Lymphoma, Systemic anaplastic large cell lymphoma, Primary cutaneous anaplastic large cell lymphoma (pcALCL) and CD30-expressing mycosis fungoides (MF) comprising administering to a subject in need thereof, a therapeutically effective amount of the engineered cell expressing the polypeptide of SEQ ID NO:54 or SEQ ID NO:56.

### BRIEF DESCRIPTION OF DRAWINGS

Exemplary embodiments are illustrated in referenced figures. It is intended that the embodiments and figures disclosed herein are to be considered illustrative rather than restrictive.

FIG. 1A shows an anti-CD19 single signal mono-CAR structure CAR-1-CD19. FIG. 1B shows an anti-CD20 single signal mono-CAR structure CAR-1-CD20. FIG. 1C shows an anti-CD22 single signal mono-CAR structure CAR-1-CD22-L-H. FIG. 1D shows an anti-CD22 single signal mono-CAR structure CAR-1-CD22-H-L. FIG. 1E shows an anti-CD30 single signal mono-CAR structure CAR-1-CD30. FIG. 1F shows an anti-EpCAM single signal mono-CAR structure CAR-1-EpCAM. FIG. 1G shows an anti-B7H4 single signal mono-CAR structure CAR-1-B7H4.

FIG. 2A shows an anti-CD19 dual-signal one-target CAR structure/system with co-expression membrane-bound cytokine. FIG. 2B shows an anti-CD20 dual-signal one-target CAR structure/system with co-expression membrane-bound cytokine. FIG. 2C shows an anti-CD22 dual-signal one-target CAR structure/system with co-expression membrane-bound cytokine. FIG. 2D shows an anti-CD30 dual-signal one-target CAR structure/system with co-expression membrane-bound cytokine. FIG. 2E shows an anti-EpCAM dual-signal one-target CAR structure/system with co-expression membrane-bound cytokine. FIG. 2F shows an anti-B7H4 dual-signal one-target CAR structure/system with co-expression membrane-bound cytokine. FIG. 2G shows an anti-MUC1 dual-signal one-target CAR structure/system with co-expression membrane-bound cytokine. FIG. 2H shows an anti-CS1 dual-signal one-target CAR structure/system with co-expression membrane-bound cytokine. FIG. 2I shows an anti-CLDN 18.2 dual-signal one-target CAR structure/system with co-expression membrane-bound cytokine. FIG. 2J shows an anti-GPC3 dual-signal one-target CAR structure/system with co-expression membrane-bound cytokine. FIG. 2K shows an anti-Mesothelin dual-signal one-target CAR structure/system with co-expression membrane-bound cytokine. FIG. 2L shows an anti-BCMA dual-signal one-target CAR structure/system with co-expression membrane-bound cytokine

FIG. 3 shows a lentiviral constructs of CAR-Ts with mbIL7, lentiviral transfer plasmid encoding scFv against human CD19 were synthesized and inserted in frame with ICOS transmembrane domain and intracellular domain and CD3zeta to create second generation CARs. Membrane bound (mb) IL-7 was generated using extracellular domain of IL7 linked by CD8 hinge region to CD8 transmembrane domain which inserted downstream of T2A to form plasmid PC035

FIG. 4 Show In vitro cell killing assay of CD19 CAR-mbIL7. After 6 rounds of co-culture of single signal CD19 CAR-T and Raji cells, the target cell killing activity was lost, however, the CD19 CAR-mbIL7 T cells remain very active to kill target cells as good as initial rounds of co-cultures, indicating CD19 CAR-mbIL7 is superior to CD19 CAR to maintain the target killing activity for longer time.

FIG. 5 shows in vivo study of CD19-mbIL-7 in lymphoma model.

### DETAILED DESCRIPTION OF THE INVENTION

The following embodiments and aspects thereof are described and illustrated in conjunction with systems, compositions and methods which are meant to be exemplary and illustrative, not limiting in scope.

Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

The term “antibody”, as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab), as well as single chain antibodies and humanized antibodies (Harlow et al., 1999. In: Using Antibodies: A Laboratory Manual, Cold Spring Harbor Laboratory Press, NY: Harlow et al., 1989, In: Antibodies: A Laboratory Manual, Cold Spring Harbor, N.Y.: Houston et al., 1988, Proc. Natl. Acad. Sci. USA 85:5879 5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene’ at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous’ is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “antigen of cancer cells” or “tumor associated antigen” as used herein is defined as a cancer biomarker selected from a group consisting of Methothelin, Muc 16, Claudin 18.2, Claudin 8, NY-ESO-1, CD 19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAX5, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR).

“Encoding” refers to the inherent property of specific sequences of nucleotides in a polynucleotide. Such as a gene, a cDNA, or an mRNA, to serve as templates for Synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous’ refers to the sequence similarity or sequence identity between two polypeptides or between two nucleic acid molecules. When a position in both of the two compared sequences is occupied by the same base or amino acid monomer Subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Co-stimulatory ligand”, as the term is used herein, includes a molecule on an antigen presenting cell (e.g., an APC, dendritic cell, B cell, and other immune cells) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7 LIGHT, NKG2C, B7-H3, and a ligand that specifically binds with CD83.

A “co-stimulatory molecule” or “co-stimulatory receptor” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA, a Toll ligand receptor. Co-stimulatory molecules also include non-natural engineered proteins.

A “co-stimulatory signal’, as used herein, refers to a signal, which in combination with a primary signal, such as TCR/CD3 ligation, leads to T cell proliferation and/or upregulation or down regulation of key molecules.

By the term “stimulation” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-B, and/or reorganization of cytoskeletal structures, and the like.

A “stimulatory molecule” as the term is used herein, means a molecule on a T cell that specifically binds with a cognate stimulatory ligand present on an antigen presenting cell.

A “stimulatory ligand”, as used herein, means a ligand that when present on an antigen presenting cell (e.g., an APC, a dendritic cell, a B-cell, and the like) can specifically bind with a cognate binding partner (referred to herein as a “stimulatory molecule’) on a T cell, thereby mediating a primary response by the T cell, including, but not limited to, activation, initiation of an immune response, proliferation, and the like. Stimulatory ligands are well-known in the art and encompass, inter cilia, an MHC Class I molecule loaded with a peptide, an anti-CD3 antibody, a Superagonist anti-CD28 antibody, and a Superagonist anti-CD2 antibody.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentivirus, adenoviral vectors, adeno-associated virus vectors, retroviral vectors, and the like. Examples of non-viral vectors include, but are not limited to CRISPR vector systems, Sleeping Beauty transposon system and the like.

“Activation”, as used herein, refers to the state of a T cell that has been sufficiently stimulated to induce detectable cellular proliferation. Activation can also be associated with induced cytokine production, and detectable effector functions. The term “activated T cells’ refers to, among other things, T cells that are undergoing cell division.

As used herein, the terms “peptide”, “polypeptide”, and “protein’ are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types, “Polypeptides’ include, for example, biologically active fragments, Substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. The term “gene” refers to a sequence of DNA or RNA which codes for a molecule that has a function.

The term “binding protein” includes natural protein binding domains (such as cytokine, cytokine receptors), antibody fragments (such as Fab, scFv, diabody, variable domain derived binders, VHH nanobody), alternative scaffold derived protein binding domains (such as Fn3 variants, ankyrin repeat variants, centyrin variants, avimers, affibody) or any protein recognizing specific antigens.

“Signal peptide”. The co-stimulatory molecule or CAR of the present invention may comprise a signal peptide so that when the co-stimulatory molecule or CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that have a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule.

The present invention provides compositions and methods for treating cancer among other diseases. Cancer may be a hematological malignancy, a solid tumor, a primary or a metastasizing tumor.

In one embodiment, an isolated polynucleotide of this invention comprises a first gene encoding a first polypeptide, wherein the first polypeptide comprises, a first antigen binding domain, a hinge domain, a transmembrane domain, a costimulatory signaling region of ICOS, and a CD3-zeta signaling domain, and a second gene encoding a second polypeptide, wherein the second polypeptide comprises a second antigen binding domain, a hinge domain and a transmembrane domain; wherein the first and second antigen binding domain binds to different antigens on cancer cells. In preferred embodiments, the polynucleotide further comprises a third nucleic acid sequence encoding a 2A peptide to link the two genes. In preferred embodiments, the first and second genes further comprise nucleic acid sequences encoding signal peptides.

In another embodiment, an isolated polynucleotide of this invention comprises a first gene encoding a first polypeptide, wherein the first polypeptide comprises an antigen binding domain, a hinge domain, a transmembrane domain, a costimulatory signaling region of ICOS, and a CD3-zeta signaling domain, and a second gene encoding a second polypeptide, wherein the second polypeptide comprises a cytokine or extracellular domain of a cytokine receptor, a hinge domain and a transmembrane domain. In preferred embodiments the polynucleotide further comprises a third nucleic acid sequence encoding a 2A peptide to link the two genes. In preferred embodiments, the first and second genes further comprise nucleic acid sequences encoding signal peptides.

In yet another embodiment, an isolated polynucleotide of this invention comprises a first gene encoding a first polypeptide and a second gene encoding a second polypeptide; wherein the first polypeptide comprises a cytokine or extracellular domain of a cytokine receptor, a hinge domain, a transmembrane domain, a costimulatory signaling region of ICOS, and a CD3-zeta signaling domain; and wherein the second polypeptide comprises an antigen binding domain, a hinge domain and a transmembrane domain. In preferred embodiments, the polynucleotide further comprises a third nucleic acid sequence encoding a 2A peptide to link the two genes. In preferred embodiments, the first and second genes further comprise nucleic acid sequences encoding signal peptides.

In another embodiment, the above polynucleotides are linked to an inducible suicide gene.

In yet another embodiment, the inducible suicide gene is linked to the above polynucleotide by a nucleic acid sequence encoding a 2A peptide.

In some embodiments, said first polypeptide contains antigen recognition domain targeting against target selected from a group consisting of Methothelin, Muc 16, Claudin 18.2, Claudin 8, NY-ESO-1, CD19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAXS, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR); and said second polypeptide contains cytokine selected from group of membrane bound IL-7, IL-21, IL-15 IL-12, IL-2, and IL-17 or extracellular domain of cytokine receptor selected from group of TGFb Receptor and IL15Ra sushi domain for treat lymphoma, leukemia and various solid tumors originated from lung, breast, prostate, colon, kidney, ovary, head and neck, liver, pancreas, bile duct and brain.

In some embodiments, said second polypeptide contains antigen recognition domain targeting against target selected from a group consisting of Methothelin, Muc 16, Claudin 18.2, Claudin 8, NY-ESO-1, CD19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAX5, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR); and said first polypeptide contains cytokine selected from group of membrane bound IL-7, IL-21, IL-15 IL-12, IL-2, and IL-17 or extracellular domain of cytokine receptor selected from group of TGFb Receptor and IL15Ra sushi domain for treat lymphoma, leukemia and various solid tumors originated from lung, breast, prostate, colon, kidney, ovary, head and neck, liver, pancreas, bile duct and brain.

In some embodiments, the engineered cell is a T-cell (CD4 and CD8 T cell) or NK cell (NKT and NK92 cell)

In some embodiments, the engineered cell comprising polynucleotides encoding dual CARs can be used for treating B-cell lymphoma and leukemia, wherein one CAR contains antigen recognition domain targeting CD19; and the other CAR contains antigen recognition domain targeting CD20 or CD22.

In some embodiments, the engineered cell comprising polynucleotides encoding dual CARs can be used for treating multiple myeloma, wherein one CAR contains antigen recognition domain targeting BCMA; and the other CAR contains antigen recognition domain targeting CD38, CD138, or CS1.

In some embodiments, the engineered cell comprising polynucleotides encoding dual CARs can be used for treating myeloid leukemia, wherein one CAR contains antigen recognition domain targeting CD123; and the other CAR contains antigen recognition domain targeting CD33 and CLL1.

In some embodiments, the engineered cell comprising polynucleotides encoding dual CARs can be used for treating prostate cancer wherein one CAR contains antigen recognition domain targeting PSCA; and the other CAR contains antigen recognition domain targeting PSMA.

In some embodiments, antigen binding domain is a scFv or a VHH nanobody.

In some embodiments, said engineered cell comprises inactivated gene of PD-1, TIM3, or LAG3 by gene knockout method.

In some embodiments, the engineered cell is an engineered T-cell or an engineered NK cell.

In some embodiments, said engineered T cell is a CD4 T-cell or CD8 T-cell.

In some embodiments, said engineered NK cell is an NKT cell or NK-92 cell.

In some embodiments, said isolated polynucleotide comprise a sequence selected from a group consisting of SEQ ID NO:6, 29, 31, 35, 37, 39, 41, 43, 45, 47, 49, and 51. In some embodiments, said polynucleotide has at least 80%, 90%, 95%, 96%, 97%, 98% or 99% identity compared with a sequence selected from a group consisting of SEQ ID NO:6, 29, 31, 35, 37, 39, 41, 43, 45, 47, 49, and 51.

In some embodiments, said isolated polynucleotide encodes a polypeptide with a sequence selected from a group consisting of SEQ ID NO:20, 30, 32, 36, 38, 40, 42, 44, 46, 48, 50, and 52. In some embodiments, said polypeptide has at least 80%, 90%, 95%, 96%, 97%, 98% or 99% identity compared with a sequence selected from a group consisting of SEQ ID NO:20, 30, 32, 36, 38, 40, 42, 44, 46, 48, 50, and 52.

In some embodiments, T cells can be transduced with a lentivirus vector to express multi-signaling chimeric antigen receptor (CAR) system with or without membrane bound cytokine/cytokine receptor fusion protein, wherein the lentivirus vector comprises an isolated polynucleotide encoding a plural of polypeptides selected from a group consisting of a polypeptide A and polypeptide B, wherein polypeptide A comprises five or more following: (i) a signal peptide, (ii) a first binding protein, (iii) a hinge region, (iv) a transmembrane domain, (v) a co-stimulatory domain consisting of ICOS and a (vi) a TCR subunit derived from CD3-zeta signaling domain, and combination thereof; and wherein polypeptide B comprises five or more following: (i) a signal peptide, (ii) a second binding protein, (iii) a hinge region, (iv) a transmembrane domain, and combination thereof. The hinge regions are optional in some embodiments.

In some embodiments, the vector is a viral vector selected from a group consisting of adenoviral vectors, adeno-associated virus vectors and retroviral vectors. In some other embodiments, the vector is a non-viral vector selected from a group consisting of CRISPR vector systems and Sleeping Beauty transposon system.

In some embodiments, genes encoding said plural of polypeptide subunits can be linked into single vector construct using a 2A peptide gene, including T2A, P2A, E2A, or F2A.

In yet another embodiment of the invention, an engineered immune cell comprises isolated polynucleotide molecule encoding an engineered membrane-bound cytokine or cytokine receptor, wherein the engineered membrane-bound cytokine or cytokine receptor comprises (i) a signal peptide, (ii) immuno-regulatory cytokines or extracellular domain of cytokine receptors, (iii) a hinge domain, (iv) a transmembrane domain, (v) a co-stimulatory domain of ICOS and a (vi) a CD3-zeta signaling domain.

In some embodiments, the cytokine is IL-7, wherein the engineered T cells producing co-stimulatory signals from membrane-bound IL-7 (mbIL-7) based co-stimulatory molecules have long-term persistence and superior in vivo antitumor activity, but not aberrant T-cell proliferation. T cells with combining expression of mblL-7 based co-stimulatory molecules with CAR retains memory potential with TSCM-like phenotype.

In yet another embodiment, an engineered immune cell comprises a polynucleotide comprising a first gene encoding a first polypeptide and a second gene encoding a second polypeptide, wherein said first polypeptide comprising (i) a signal peptide, (ii) a binding protein, (iii) a hinge region, (iv) a transmembrane domain, (v) a co-stimulatory signaling domain of ICOS and (vi) a CD3 zeta signaling domain; and said second polypeptide comprising (i) a signal peptide, (ii) an immuno-regulatory cytokines or extracellular domain of cytokine receptors, (iii) a hinge region, (iv) a transmembrane domain; wherein the binding protein binds to an antigen on cancer cells; wherein the first gene and second gene are linked by a gene encoding a 2A peptide.

In preferred embodiments, the present invention provides a single vector expressing two chimeric antigen receptors (CARs), or one CAR and one other co-stimulatory molecule each comprising an extracellular and intracellular domain. The extracellular domain of a CAR comprises a target-specific binding element otherwise referred to as an antigen binding moiety. The extracellular domain of the other co-stimulatory molecule comprises an immune-regulatory cytokine or extracellular domain of a cytokine receptor. The intracellular domain or otherwise the cytoplasmic domain comprises, a costimulatory signaling region and a Zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to primary antigen. Between the extracellular domain and the transmembrane domain of the CAR, or between the cytoplasmic domain and the transmembrane domain of the CAR, there may be incorporated a hinge domain. As used herein, the term “hinge domain generally means any oligo- or polypeptide that functions to link the transmembrane domain to, either the extracellular domain or, the cytoplasmic domain in the polypeptide chain. A hinge domain may comprise up to 300 amino acids, preferably 10 to 100 amino acids and most preferably 25 to 50 amino acids. Hinge region (“H”) can be a single or plural of H1 (SEQ ID NO:16).

The transmembrane domain may be derived either from a natural or from a synthetic source. Where the source is natural, the domain may be derived from (i.e. comprise at least the transmembrane region(s) of) any membrane-bound or transmembrane protein such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Transmembrane regions of preferred embodiments may be derived from human-origin with the sequence of SEQ ID NOs:17 or 28. Alternatively the transmembrane domain may be synthetic, in which case it will comprise predominantly hydrophobic residues such as leucine and valine. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain Sufficient to transduce the effector function signal.

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from CD3 zeta, FcRgamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3 zeta chain (CD3-zeta) signaling domain combined with any other desired cytoplasmic domain(s) useful in the context of the CAR of the invention. For example, the cytoplasmic domain of the CAR or any other co-stimulatory molecule can comprise a CD3 Zeta chain portion and a costimulatory signaling region. The costimulatory signaling region refers to a portion of the CAR comprising the intracellular domain of a costimulatory molecule. A costimulatory molecule is a cell surface molecule other than a primary antigen receptor or their ligands that is required for an efficient response of lymphocytes to an antigen. Examples of such molecules include CD27, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. It is preferred that co-stimulatory signaling element in the CAR or co-stimulatory molecule of the invention is ICOS. And it is particularly preferred that the cytoplasmic domain comprises the combination of ICOS/CD3-zeta.

In one embodiment, multiple-costimulatory-signal CAR structures with an antigen binding domain targeting tumor-specific antigen CD19 and an antigen binding domain targeting another tumor-specific antigen including CD20 and CD22. In another embodiment, the multiple-costimulatory-signal CAR structure contains a membrane-bound cytokine and an antibody targeting tumor-specific antigens including CD19, CD20, CD22, CD30, EpCAM, MUC-1, CS2, CLDN 18.2, GPC3, Mesothelin, BCMA and B7H4. In one embodiment, the membrane-bound cytokine of CAR structures is mbIL-7.

In another embodiment, the multiple-costimulatory-signal CAR T cells further contain inducible suicide gene.

Strategies for multigene co-expression with a single vector include use of multiple promoters, fusion proteins, proteolytic cleavage sites between genes, internal ribosome entry sites, and “self-cleaving” 2A peptides. 2A peptides are 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was recently discovered to be ribosome skipping the formation of a glycyl-prolyl peptide bond at the C-terminus of the 2A. (Donnelly, M. L. et al, 2001).

The DNA encoding the new CAR system were synthesized and cloned into lentiviral vectors. Those vector plasmids will be manufactured with quality control in 293 T cells into mature lentiviral particles and the T cells or other immune cells such NK or NKT cells isolated from patient's PBMC will be transduced with lentivirus containing our new CAR structure. The transduced immune cells will grow and expand in bioreactor about 10 days to reach therapeutic number. After quality control release, these CAR-expressing immune cells will be transfused back to patients for medical use.

Compositions, Formulations and Methods of Administration

Also provided are cells, cell populations, and compositions (including pharmaceutical and therapeutic compositions) containing the cells and populations, such as cells and populations produced by the provided methods. Also provided are methods, e.g., therapeutic methods for administrating the cells and compositions to subjects, e.g., patients.

Compositions and Formulations

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

Methods of Administration and Uses of Cells in Adoptive Cell Therapy

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the antigen associated with the disease or disorder is selected from the group consisting of Methothelin, Muc 16, Claudin 18.2, Claudin 8, NY-ESO-1, CD19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAXS, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR) and/or biotinylated molecules, and/or molecules expressed by HIV, HCV, HBV or other pathogens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

Dosing Schedule or Regimen

In some embodiments, repeated dosage methods are provided in which a first dose of cells is given followed by one or more second consecutive doses. The timing and size of the multiple doses of cells generally are designed to increase the efficacy and/or activity and/or function of antigen-expressing T cells, such as CAR-expressing T cells, when administered to a subject in adoptive therapy methods. In some embodiments, the repeated dosings reduce the downregulation or inhibiting activity that can occur when inhibitory immune molecules, such as PD-1 and/or PD-L1 are upregulated on antigen-expressing, such as CAR-expressing, T cells. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first or consecutive dose is a single or continuous administration of the specified number of cells, given or initiated at a single point in time. In some contexts, however, the first or consecutive dose is administered in multiple injections or infusions over a period of no more than three days, such as once a day for three days or for two days or by multiple infusions over a single day period.

Thus, in some aspects, the cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses may be administered in a plurality of compositions over a period of no more than 3 days.

The term “split dose” refers to a dose that is split so that it is administered over more than one day. This type of dosing is encompassed by the present methods and is considered to be a single dose.

Thus, the first dose and/or consecutive dose(s) in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject received a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses, with each consecutive dose given within a period of time in which an inhibitory immune molecule, such as PD-1 and/or PD-L1, has been upregulated in cells in the subject from an administered first dose. It is within the level of a skilled artisan to empirically determine when to provide a consecutive dose, such as by assessing levels of PD-1 and/or PD-L1 in antigen-expressing, such as CAR-expressing cells, from peripheral blood or other bodily fluid.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg, each inclusive.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, the receptor, e.g., the CAR, expressed by the cells in the consecutive dose contains at least one immunoreactive epitope as the receptor, e.g., the CAR, expressed by the cells of the first dose. In some aspects, the receptor, e.g., the CAR, expressed by the cells administered in the consecutive dose is identical to the receptor, e.g., the CAR, expressed by the first dose or is substantially identical to the receptor, e.g., the CAR, expressed by the cells of administered in the first dose.

The recombinant receptors, such as CARs, expressed by the cells administered to the subject in the various doses generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells in the first dose express a CAR that specifically binds to an antigen expressed by a cell or tissue of the disease or condition or associated with the disease or condition.

TABLE 1
Representative Sequences identified for polynucleotide of CAR-structure/system
and domains of CARs

| IDENTITY | SEQ ID NO: # |
|---|---|
| SP1 (gm-sp) | (SEQ ID NO: 1) |
| hinge 1 (CD8a hinge) | (SEQ ID NO: 2) |
| TM1 | (SEQ ID NO: 3) |
| ICOS intracellular domain (ICOS) | (SEQ ID NO: 4) |
| CD3zeta | (SEQ ID NO: 5) |
| Signal1 structure: TM1 + ICOS + CD3zeta | (SEQ ID NO: 6) |
| T2A | (SEQ ID NO: 7) |
| P2A | (SEQ ID NO: 8) |
| E2A | (SEQ ID NO: 9) |
| F2A | (SEQ ID NO: 10) |
| SP2 | (SEQ ID NO: 11) |
| IL-7 | (SEQ ID NO: 12) |
| hinge2 (H2) | (SEQ ID NO: 13) |
| TM2 | (SEQ ID NO: 14) |
| Full humanized anti-CD19 single CAR (CAR-1-CD19) | SEQ ID NO: 29 |
| Full humanized anti-CD19/mb-IL-7 dual signal CAR (CAR-1- | SEQ ID NO: 31 |
| CD19/CAR-2-IL-7) | |
| SP3 | SEQ ID NO: 33 |
| Full humanized anti-CD19/anti-CD20 dual signal and dual CAR | SEQ ID NO: 35 |
| (CAR-1-CD19/CAR-2-CD20) | |
| Full humanized anti-CD20 single CAR (CAR-1-CD20) | SEQ ID NO: 37 |
| Full humanized anti-CD22(L-H) single CAR (CAR-1-CD22) | SEQ ID NO: 39 |
| Full humanized anti-CD20(H-L) single CAR (CAR-1-CD22 | SEQ ID NO: 41 |
| (H/L)) | |
| Full humanized anti-CD20/mb-IL-7 dual-signal CAR (CAR-1- | SEQ ID NO: 43 |
| CD20/CAR-2-IL-7) | |
| Full humanized anti-CD22(L-H)/mb-IL-7 dual-signal CAR | SEQ ID NO: 45 |
| (CAR-1-CD22 (L/H)/CAR-2-IL-7) | |
| Full humanized anti-CD22(H-L)/mb-IL-7 dual-signal CAR | SEQ ID NO: 47 |
| (CAR-1-CD22 (H/L)/CAR-2-IL-7) | |
| Full humanized anti-CD19/anti-CD22 (L-H) dual-signal and dual | SEQ ID NO: 49 |
| CAR (CAR-1-CD19/CAR-2-CD22 (L/H)) | |
| Full humanized anti-CD19/anti-CD22 (H-L) dual-signal and dual | SEQ ID NO: 51 |
| CAR (CAR-1-CD19/CAR-2-CD22(H/L)) | |
| Full humanized anti-CD30 single CAR (CAR-1-CD30) | SEQ ID NO: 53 |
| Full anti-CD30/mb-IL-7 dual signal CAR (CAR-1-CD30/CAR-2- | SEQ ID NO: 55 |
| IL-7) | |

TABLE 2
Representative sequences identified for protein/peptide encoded by
polynucleotides of CAR-structure/system and domains of CARs

| IDENTITY | SEQ ID NO: # |
|---|---|
| SP1 (gm-sp) | SEQ ID NO: 15 |
| hinge 1 (CD8a hinge) | SEQ ID NO: 16 |
| TM1 | SEQ ID NO: 17 |
| ICOS intracellular domain (ICOS) | SEQ ID NO: 18 |
| CD3zeta | SEQ ID NO: 19 |
| Signall structure: TM1 + ICOS + CD3zeta | SEQ ID NO: 20 |
| T2A | SEQ ID NO: 21 |
| P2A | SEQ ID NO: 22 |
| E2A | SEQ ID NO: 23 |
| F2A | SEQ ID NO: 24 |
| SP2 | SEQ ID NO: 25 |
| IL-7 | SEQ ID NO: 26 |
| hinge2 (H2) | SEQ ID NO: 27 |
| TM2 | SEQ ID NO: 28 |
| Full humanized anti-CD19 single CAR (CAR-1-CD19) | SEQ ID NO: 30 |
| Full humanized anti-CD19/mb-IL-7 dual-signal CAR (CAR-1- | SEQ ID NO: 32 |
| CD19/CAR-2-IL-7) | |
| SP3 | SEQ ID NO: 34 |
| Full humanized anti-CD19/anti-CD20 dual-signal and dual | SEQ ID NO: 36 |
| CAR (CAR-1-CD19/CAR-2-CD20) | |
| Full humanized anti-CD20 single CAR (CAR-1-CD20) | SEQ ID NO: 38 |
| Full humanized anti-CD22(L-H) single CAR (CAR-1-CD22 | SEQ ID NO: 40 |
| (L/H)) | |
| Full humanized anti-CD20(H-L) single CAR (CAR-1-CD22 | SEQ ID NO: 42 |
| (H/L)) | |
| Full humanized anti-CD20/mb-IL-7 dual-signal CAR (CAR-1- | SEQ ID NO: 44 |
| CD20/CAR-2-IL-7) | |
| Full humanized anti-CD22(L-H)/mb-IL-7 dual-signal CAR | SEQ ID NO: 46 |
| (CAR-1-CD22 (L/H)/CAR-2-IL-7) | |
| Full humanized anti-CD22(H-L)/mb-IL-7 dual-signal CAR | SEQ ID NO: 48 |
| (CAR-1-CD22 (H/L)/CAR-2-IL-7) | |
| Full humanized anti-CD19/anti-CD22 (L-H) dual-signal and | SEQ ID NO: 50 |
| dual CAR (CAR-1-CD19/CAR-2-CD22) | |
| Full humanized anti-CD19/anti-CD22 (H-L) dual-signal and | SEQ ID NO: 52 |
| dual CAR (CAR-1-CD19/CAR-2-CD22(H/L)) | |
| Full humanized anti-CD30 single CAR (CAR-1-CD30) | SEQ ID NO: 54 |
| Full anti-CD30/mb-IL-7 dual signal CAR (CAR-1-CD30/CAR- | SEQ ID NO: 56 |
| 2-IL-7) | |

TABLE 3
Expression level of CAR and mbIL-7

| Cells | Protein L | CD19 antigen | anti-IL-7 antibody |
|---|---|---|---|
| CD19 CAR-mIL7 T cells | 49% | 42% | 25% |

### WORKING EXAMPLES:

### Example 1

### Preparation of Single-Signal Mono-CAR

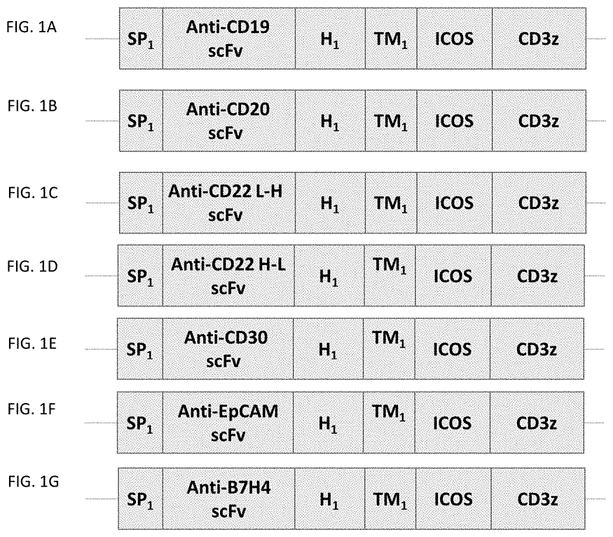

A CAR backbone sequence encoding a CAR backbone polypeptide comprising from the N-terminus to the C-terminus: a CD8 hinge domain, an ICOS transmembrane domain, an ICOS cytoplasmic domain and a CD3zeta cytoplasmic domain were chemically synthesized and cloned into a pre-modified lentiviral transfer vector downstream and operably linked to a constitutive hEF1a promotor. DNA sequence and peptide sequence of SP1 (gm-sp) are SEQ ID NOs:1 and 15 respectively; DNA sequence and peptide sequence of H1 (hinge 1) are SEQ ID NOs:2 and 16 respectively; DNA sequence and peptide sequence of TM1 are SEQ ID NOs: 3 and 17 respectively (Tables 1 and 2). The resulting CAR1 backbone vector was named pAC_C01. EcoRI site in the vector allowed insertion of a nucleic acid sequence comprising a Kozak sequence operably linked to a nucleic acid sequence encoding a GM-CSF signal peptide fused to the N-terminus of scFv of humanized anti-CD19 (#Ab01), humanized anti-CD20(#Ab02, humanized anti-CD22 (L-H and H-L two forms, #Ab03 and ##Ab04 respectively), humanized anti-CD30 (#Ab05), humanized anti-EpCAM (#Ab06) and humanized anti-B7H4 (#Ab07) fragments into the pAC_CO1 vector by sequence and ligation independent cloning method (SLIC), upstream and operably linked to the CD8 hinge domain of CAR-1 backbone sequence (FIG. 1A-1G). The polynucleotide of single signal mono-CAR structure is named as CAR-1-CD19 (SEQ ID NO:29), CAR-1-CD20 (SEQ ID NO:37), CAR-1-CD22 (L/H) (SEQ ID NO:39), CAR-1-CD22 (H/L) (SEQ ID NO:41), CAR-1-CD30 (SEQ ID NO:53), CAR-1-EpCAM, and CAR-1-B7H4, respectively (Table 1). The polypeptide encoded by the polynucleotides of Table 1 are shown Table 2. The vector construct of single signal mono-CAR is named as pCAR-1-CD19, pCAR-1-CD20, pCAR-1-CD22 (L/H), pCAR-1-CD22 (H/L), pCAR-1-CD30, pCAR-1-EpCAM, and pCAR-1-B7H4, respectively.

### Example 2

### Preparation of Dual-Signal Mono-CAR

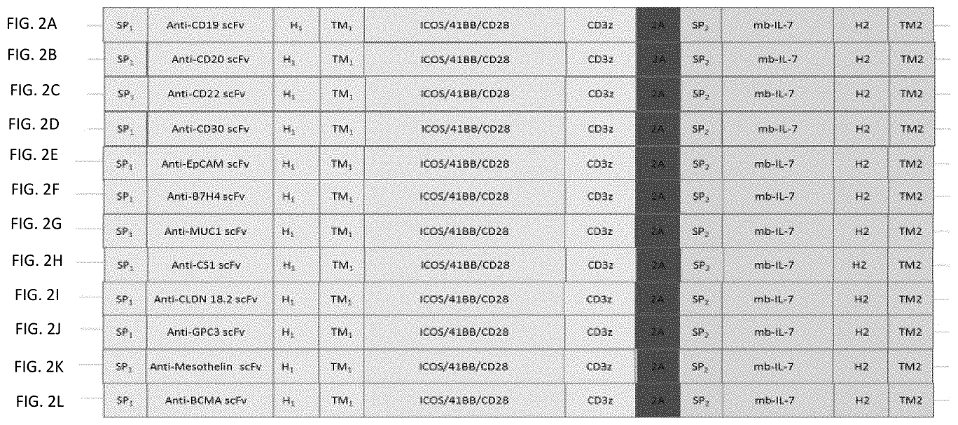

A 2nd CAR backbone sequence is encoding a polypeptide comprising from the N-terminus to the C-terminus: a modified CD8 hinge domain, a CD137 transmembrane domain, a CD137 cytoplasmic domain and a CD3e cytoplasmic domain were chemically synthesized and cloned into pAC_CO1 and named as pAC_C0102. DNA sequence and peptide sequence of SP1 (gm-sp) are SEQ ID NOs:1 and 15 respectively; DNA sequence and peptide sequence of SP2 are SEQ ID NOs:11 and 25 respectively; DNA sequence and peptide sequence of H1 (hinge 1) are SEQ ID NOs:2 and 16 respectively; DNA sequence and peptide sequence of H2 (hinge 2) are SEQ ID NOs:13 and 27 respectively; DNA sequence and peptide sequence of TM2 are SEQ ID NOs:14 and 28 respectively; DNA sequence and peptide sequence of T2A are SEQ ID NOs:7 and 21 respectively (Table 1 and 2). DNA sequence and peptide sequence of Signall structure (TM1+ICOS+CD3zeta) are SEQ ID NOs:6 and 20 respectively. BamHI site in the vector allowed insertion a synthetic nucleic acid sequence encoding a T2A peptide, IL-7 signal peptide, IL-7 extracellular fragments into the pCAR-1-CAR-2 vector, upstream and operably linked to the CD137 transmembrane domain of CAR-2 backbone sequence (FIG. 2A-2G). The polynucleotide of dual-signal mono-CAR structure is named as CAR-1-CD19/CAR-2-IL-7 (SEQ ID NO:31), CAR-1-CD20/CAR-2-IL-7 (SEQ ID NO:43), CAR-1-CD22 (L/H)/CAR-2-IL-7(SEQ ID NO:45), CAR-1-CD22 (H/L)/CAR-2-IL-7 (SEQ ID NO:47), CAR-1-CD30/CAR-2-IL-7 (SEQ ID NO:55), CAR-1-EpCAM/CAR-2-IL-7, and CAR-1-B7H4/CAR-2-IL-7, respectively (Table 1). The polypeptides encoded by the polynucleotides of Table 1 are shown Table 2. The vector construct of dual-signal mono-CAR is named as pCAR-1-CD19/CAR-2-IL-7, pCAR-1-CD20/CAR-2-IL-7, pCAR-1-CD22 (L/H)/CAR-2-IL-7, pCAR-1-CD22 (H/L)/CAR-2-IL-7, CAR-1-CD30/CAR-2-IL-7, pCAR-1-EpCAM/CAR-2-IL-7, and pCAR-1-B7H4/CAR-2-IL-7, respectively.

### Example 3

### Preparation of Dual-Signal Dual Targeting-CAR

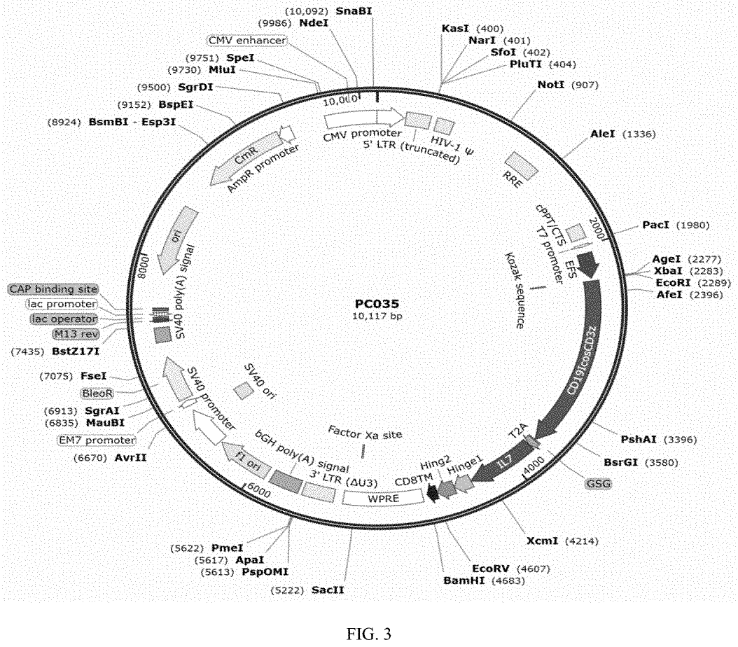

A 2nd CAR backbone sequence encoding a polypeptide comprising from the N-terminus to the C-terminus: a CD8 hinge domain, a CD137 transmembrane domain, a CD137 cytoplasmic domain and a CD3e cytoplasmic domain were chemically synthesized and cloned into pCAR-1-CD19 by SLIC and named as pCAR-1-CD19/CAR-2. DNA sequence and peptide sequence of SP1 (gm-sp) are SEQ ID NOs:1 and 15 respectively; DNA sequence and peptide sequence of SP3 are SEQ ID NOs: 33 and 34 respectively; DNA sequence and peptide sequence of H1 (hinge 1) are SEQ ID NOs:2 and 16 respectively; DNA sequence and peptide sequence of H2 (hinge 2) are SEQ ID NOs:13 and 27 respectively; DNA sequence and peptide sequence of TM2 are SEQ ID NOs:14 and 28; DNA sequence and peptide sequence of T2A are SEQ ID NOs:7 and 21 respectively. DNA sequence and peptide sequence of Signall structure (TM1+ICOS+CD3zeta) are SEQ ID NO:6 and 20 respectively. ACC65I site in the vector allowed insertion of nucleic acid sequence encoding a T2A peptide, a synthetic nucleic acid sequence encoding a CD8 signal peptide fused to the N-terminus of scFv of humanized anti-CD20, humanized anti-CD22 into the pCAR-1-CD19/CAR-2 vector, upstream and operably linked to the CD8 hinge domain of CAR-2 backbone sequence (FIG. 3A-3C). The polynucleotide of dual-signal dual-CAR structure is named as CAR-1-CD19/CAR-2-CD20 (SEQ ID NO:35), CAR-1-CD19/CAR-2-CD22 (L/H) (SEQ ID NO:49), and CAR-1-CD19/CAR-2-CD22(H/L) (SEQ ID NO:51), respectively (Table 1). The polypeptide encoded by the polynucleotides of Table 1 are shown Table 2. The vector construct of dual-signal dual-CAR is named as pCAR-1-CD19/CAR-2-CD20, pCAR-1-CD19/CAR-2-CD22 (L/H), and pCAR-1-CD19/CAR-2-CD22(H/L), respectively.

### Example 2

### Lentiviral Construct: and Lentivirus Production

A third generation of lentiviral transfer plasmid encoding scFv against human CD19 were synthesized and inserted in frame with ICOS transmembrane domain and intracellular domain and CD3zeta to create second generation CARs. Membrane bound (mb) IL-7 was generated using extracellular domain of IL7 linked by CD8 hinge region to CD8 transmembrane domain which inserted downstream of T2A to form plasmid PC035 (FIG. 3). To produce lentivirus for transduction of T cells, 293T cells were transfected with PC035 encoding CD19 CAR-mIL7 together with packaging plasmids PCO26, PCO27,PCO28. After concentration and purification, the physical titer of lentivirus was measured by ELISA measurement of p24 level. 2×108 TU can be achieved through this procedure.

### Example 3

### CD19 CAR-mbIL7 Expression on T Cells

T cells from donors were transduced using lentivirus encoding CD19 CAR-mIL7. After 24 hours, the positivity of CD19CAR and membrane bound (mb) IL7 were detected by flow cytometer using Protein L, CD19 protein and anti-IL7 antibody, respectively. The expression level was shown in Table 3.

### Example 4

### In Vitro Target Cell Killing Assay of CD19 CAR-mbIL7

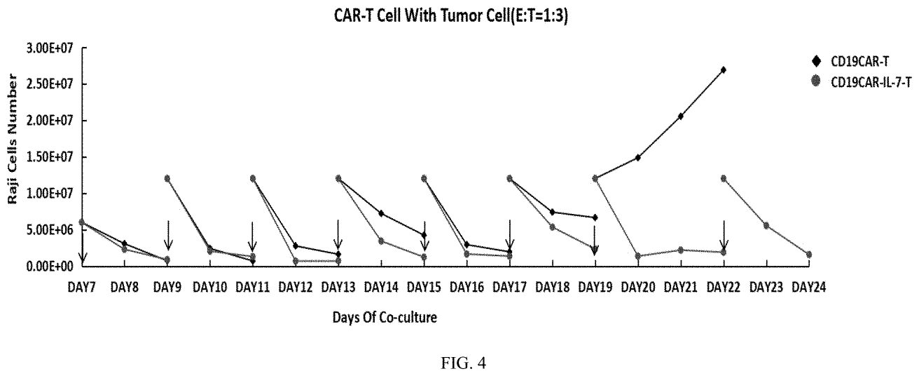

CD19 CAR-mbIL7 transduced T cells were co-cultured for with Raji cells (E/T ratio=1/3) for 3 days, the target cell killing was measured using Promega cell viability kit. After cell killing measurement, CD19 CAR-mbIL7 transduced T cells were isolated out from coculture system and remixed with fresh Raji cells (E/T ratio=1/3) and continue culture for 3 days for next round of cell killing measurement. These procedures were repeated until T cells lost the killing activity to target cells. The result is shown in FIG. 4.

### Example 7

### Cytotoxic Assay of CD20-CAR

Target cells pre-labeled with CFSE (K562, Raji cells or Nalm-6 cells) seeded at 5 ×10⁴ cells/well in a 96-well plate and co-incubated with effector cells (CAR-CD20 transduced T cells or non-transduced T cell as control) at varying E:T ratios in complete OpTmizer™ CTS™ T-Cell Expansion Basal Medium for 4 hours. A flow cytometry-based cytotoxicity assay was established by gating out of the CFSE positive population and detecting using Annexin on APC channel and PI on PE channel. The test results showed that the cytotoxicity of CD20-CAR is specific to CD20 positive cells (FIG. 6) and more than 80% target cell killing at E/T ratio at 10:1 was achieved in both Raji and Nalm-6 co-culture system.

### Example 8

### Cytotoxic Assay of CD22-CAR

Target cells pre-labeled with CFSE (K562, Raji cells or Nalm-6 cells) seeded at 5 ×10⁴ cells/well in a 96-well plate and co-incubated with effector cells (CAR-CD22 transduced T cells or non-transduced T cell as control) at varying E:T ratios in complete OpTmizer™ CTS™ T-Cell Expansion Basal Medium for 4 hours. A flow cytometry-based cytotoxicity assay was established by gating out of the CFSE positive population and detecting using Annexin on APC channel and PI on PE channel. The test results showed that the cytotoxicity of CD22-CAR is specific to CD22 positive cells (FIG. 7) and 60% cell killing activity in Nalm-6 and 40% cell killing activity in Raji co-culture system were achieved.

### Example 9

### Cytotoxic Assay of Dual-Signal CARs

Target cells pre-labeled with CFSE (Raji cells or Nalm-6 cells) seeded at 5 ×10⁴ cells/well in a 96-well plate and co-incubated with effector cells (CAR-CD19/CD20 transduced T cells, CAR-CD19/CD22 and CAR-19-mbIL-7 transduced T cells) at varying E:T ratios in complete OpTmizer™ CTS™ T-Cell Expansion Basal Medium for 4 hours. A flow cytometry-based cytotoxicity assay was established by gating out of the CFSE positive population and detecting using Annexin on APC channel and PI on PE channel. CAR-19-20 and CAR-19-22 achieved 80% cell killing at a E/T ratio at 10:1. Car-19-mbIL-17 achieved 90% cell killing activity in Raji co-culture assay (data not shown) at a E/T ratio at 10:1.

### Example 10

### Cytokine Production Quantification

Target cells (Raji and Nalm-6 cells) were seeded at 5×104 cells/well in a 96-well plate and co-incubated with effector cells at various E:T ratio for 4 hours and 24 hours. Cytokine concentrations in the culture supernatant (INFgamma and IL-2) were measured by the method of TR-FRET. All CAR-T cells produced significant amount of INFgamma but not IL-2 in both Raji and Nalm-6 co-culture system. The INF gamma release was found to be correlated to E/T ratio and targeted cell killing.

### Example 11

### T Cells Differentiation After CAR-CD19 Transduction

PBMC were obtained from healthy donors and T cells were isolated, activated using Dynabeads ClinExVivo CD3/CD28 following the manufacturer's recommendations (Invitrogen). On day 2, activated T cells were transduced with Lentivirus at a multiplicity of infection (MOI) of 1.5. All T cells were expanded in complete T-cell medium supplemented with IL-2 (50 U/ml) and IL-15 (1 ng/ml). Dynabeads were removed by magnetic on day 12 before further analysis of the transduced T cells in in vitro assays. T cells subtype distribution was assessed by double staining of CD45RO and CCR7. After CAR-CD19 transduction in T cells, the effector memory T cell population (CCR7-CD45RO+) greatly increased in both CD4 and CD8 T cell subtype.

### Example 5

### In Vivo Study of CD19 CAR-mbIL7 in Lymphoma Model

A million Raji-GFP-Luc cells (from Biocytogen) were injected into the tail vein of Immune-deficient B-NDG (NOD-Prkdcscid IL2rgtml/Bcgen) mice. On day 6 following Raji-GFP-Luc injection, tumor engraftment was measured by i.p. injection of 150 mg/kg luciferin and imaged 10 min later for 180 s on a In Vivo-Xtreme imaging system (Bruker). Images were overlapped on 30 s X-ray Image, and the bioluminescent signal flux for each mouse was expressed as average radiance (photons per second per cm2 per steradian, P/S). 5×108 CAR T cells/kg of CD19-CAR mbIL7 (high dose), CD19 single CAR or 5×107 CD19-CAR mbIL7 lowdose) were administered to mice via tail vein injection on Day 6 after group randomization based on equally-distributed P/S value. Imaging was performed on days 7, 14, 28, 35,42 post treatment to establish the kinetics of tumor growth and tumor eradication by CAR T cells.

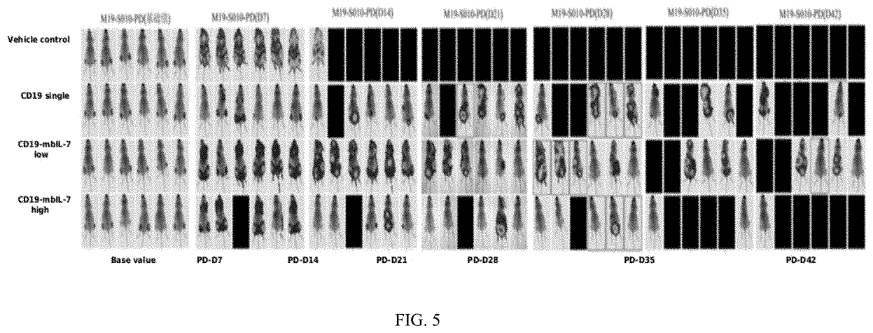

Representative images of the progression or regression of disease in each group were shown in FIG. 5. By Day 15. all animals in control group died of fast growing tumors. In animals dosed with CD19 single CAR T cells had a short term effect (about 2 weeks), and tumors eventually progressed after 3 weeks treatment and 4/6 animals died of progressed tumors by Day 42. However, CD19-mbIL-7 less dosed CAR treatment started taking effect after 3 weeks treatment and greatly slow tumor growth. By Day 42, only 1 out 6 animals died of tumor burden. Interestingly, high dose treatment of CD19-mbIL-7 took effect after 2 weeks, and significantly regressed tumor growth as compared with same dose of single CD19 CAR T cells. 4/6 animal showed elimination of tumors in high dose CD19-mbIL-7 group. This group of human T cells with high efficacy may cause GVHD effect to the tumor bearing mice and lead to animal death not caused by tumor overgrowth. This phenomenon will not happen when dosing human T cells in human patients. Although the above invention has been described in some detail by way of illustration and example for purposes of clarity of understanding, it is apparent to those skilled in the art that certain changes and modifications will be practiced. Therefore, the description and examples should not be construed as limiting the scope of the disclosed invention.

The disclosures of all publications, patents, patent applications and published patent applications referred to herein by an identifying citation are hereby incorporated herein by reference in their entirety.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### REFERENCES

Berger C, et al. Adoptive transfer of effector CD8+ T cells derived from central memory cells establishes persistent T cell memory in primates. J Clin Invest. 2008; 118(1):294-305.

Cieri N, et al. IL-7 and IL-15 instruct the generation of human memory stem T cells from naive precursors. Blood. 2013; 121(4):573-584.

Dadi S, et al. Cancer immunosurveillance by tissue-resident innate lymphoid cells and innate-like T cells. Cell. 2016; 164(3):365-377.

Donnelly, M. L. et al. The ‘cleavage’ activities of foot-and-mouth disease virus 2A site-directed mutants and naturally occurring ‘2A-like’ sequences. 2001. The Journal of general virology 82, 1027-1041.

Donnelly, M. L. et al. Analysis of the aphthovirus 2A/2B polyprotein ‘cleavage’ mechanism indicates not a proteolytic reaction, but a novel translational effect: a putative ribosomal ‘skip’. 2001. The Journal of general virology 82, 1013-1025.

Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc Natl Acad Sci USA. 1993; 90(2):720-724.

Gattinoni L, et al. A human memory T cell subset with stem cell-like properties. Nat Med. 2011; 17(10):1290-1297.

Grupp S A, et al. Chimeric antigen receptor-modified T cells for acute lymphoid leukemia. N Engl J Med. 2013 Apr. 18; 368(16):1509-1518.

Hinrichs C S, et al. Human effector CD8+ T cells derived from naive rather than memory subsets possess superior traits for adoptive immunotherapy. Blood. 2011; 117(3):808-814.

Kalos M, et al. T cells with chimeric antigen receptors have potent antitumor effects and can establish memory in patients with advanced leukemia. Sci Transl Med. 2011 Aug. 10; 3(95):95ra73.

Klebanoff C A, et al. IL-15 enhances the in vivo antitumor activity of tumor-reactive CD8+ T cells. Proc Natl Acad Sci USA. 2004; 101(7):1969-1974.

Kochenderfer J N, et al. Eradication of B-lineage cells and regression of lymphoma in a patient treated with autologous T cells genetically engineered to recognize CD19. Blood. 2010; 116(20):4099-102.

Kochenderfer J N, et al. Donor-derived CD19-targeted T cells cause regression of malignancy persisting after allogeneic hematopoietic stem cell transplantation. Blood. 2013; 122(25):4129-39.

Lugli E, et al. Superior T memory stem cell persistence supports long-lived T cell memory. J Clin Invest. 2013; 123(2):594-599.

Marks-Konczalik J, et al. IL-2-induced activation-induced cell death is inhibited in IL-15 transgenic mice. Proc Natl Acad Sci USA. 2000; 97(21):11445-11450.

Maude S L et al. Chimeric antigen receptor T cells for sustained remissions in leukemia. N Engl J Med. 2014; 371(16):1507-17.

Porter D L, et al. Chimeric antigen receptor-modified T cells in chronic lymphoid leukemia. N Engl J Med. 2011; 365(8):725-33.

Porter D L, et al. Chimeric antigen receptor T cells persist and induce sustained remissions in relapsed refractory chronic lymphocytic leukemia. Sci Transl Med. 2015; 7(303):303ra139.

Sabatino M, et al. Generation of clinical-grade CD19-specific CAR-modified CD8+ memory stem cells for the treatment of human B-cell malignancies. Blood. 2016; 128(4):519-528.

Savoldo B, et al. CD28 costimulation improves expansion and persistence of chimeric antigen receptor-modified T cells in lymphoma patients. J Clin Invest. 2011; 121(5):1822-1826.

Xu Y, et al. Closely related T-memory stem cells correlate with in vivo expansion of CAR.CD19-T cells and are preserved by IL-7 and IL-15. Blood. 2014; 123(24):3750-3759.

Zhang X, Sun S, Hwang I, Tough D F, Sprent J. Potent and selective stimulation of memory-phenotype CD8+ T cells in vivo by IL-15. Immunity. 1998; 8(5):591-599.

Teague R M, et al. Interleukin-15 rescues tolerant CD8+ T cells for use in adoptive immunotherapy of established tumors. Nat Med. 2006; 12(3):335-341.

Gruenberg et al. Re-activated T-cells for adoptive immunotherapy. US Patent Application no. US 2003,0170,238 A1.

Rosenberg et al. Adoptive immunotherapy as a treatment modality in humans. U.S. Pat. No. 4,690,915 A.

We claim:

1. An isolated polynucleotide comprising a first gene encoding a first polypeptide and a second gene encoding a second polypeptide, wherein said first polypeptide comprising five or more of the following: (i) a signal peptide, (ii) a binding protein, (iii) a hinge region, (iv) a transmembrane domain, (v) a co-stimulatory signaling domain of ICOS and (vi) a TCR CD3 zeta signaling domain;
and said second polypeptide comprising four or more of the following (i) a signal peptide, (ii) a binding protein, (iii) a hinge region, (iv) a transmembrane domain; wherein at least one of the binding protein binds to an antigen on cancer cells.

2. The polynucleotide of claim 1, wherein the binding protein of said first polypeptide is an antigen recognition domain and the binding protein of said second polypeptide is an immuno-regulatory cytokine or an extracellular domain of cytokine receptor, wherein said first polypeptide binds to an antigen on cancer cells.

3. The polynucleotide of claim 1, wherein the binding protein of said first polypeptide is an immuno-regulatory cytokine or an extracellular domain of cytokine receptor and the binding protein of said second polypeptide is an antigen recognition domain, wherein said second polypeptide binds to an antigen on cancer cells.

4. The polynucleotide of claim 1, wherein the binding protein of said first polypeptide comprises a first antigen recognition domain and the binding protein of said second polypeptide comprises a second antigen recognition domain, wherein the first and second antigen recognition domain binds different antigens on cancer cells.

5. The polynucleotide of any one of claims 1 to 4, further comprising a third gene encoding a 2A peptide; wherein said first gene and said second gene are linked by the third gene.

6. The polynucleotide of claim 1, wherein said first polypeptide comprises binding protein targeting against target selected from a group consisting of Methothelin, Muc 16, Claudin 18.2, Claudin 8, NY-ESO-1, CD19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGSS), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAXS, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR); and second polypeptide comprises binding protein selected from a group consisting of IL-7, IL-21, IL-15 IL-12, IL-2, IL-17, IL15Ra sushi domain, and extracellular domain of TGFb Receptor.

7. The polynucleotide of claim 1, wherein said second polypeptide comprises binding protein targeting against target selected from a group consisting of Methothelin, Muc 16, Claudin 18.2, Claudin 8, NY-ESO-1, CD19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), B7-H3 (CD276), B7-H4, KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), CD20, Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGSS), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAXS, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD79b, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), ganglioside GD3, signaling lymphocyte activation molecule (SLAM) family member 6 (SLAMF6), SLAMF4, Leutenizing hormone receptor (LHR), follicle stimulating hormone receptor (FSHR), and Chorionic Gonadotropin Hormone receptor (CGHR); and first polypeptide comprises binding protein selected from a group consisting of IL-7, IL-21, IL-15, IL-12, IL-2, IL-17, IL15Ra sushi domain and extracellular domain of TGFb Receptor.

8. A polypeptide comprising a peptide encoded by the first gene of claim 1.

9. A polypeptide comprising a peptide encoded by the second gene of claim 1.

10. The isolated polynucleotide according to any one of claims 2-4, wherein the antigen recognition domain is a scFv or a VHH nanobody.

11. An expression vector comprising the polynucleotide of any one of claims 1-4.

12. An engineered cell comprising an expression vector of claim 11.

13. An engineered cell of claim 12, wherein the first binding protein binds to CD19; and the second binding protein binds to CD20 or CD22.

14. An engineered cell of claims 12, wherein the first binding protein binds to BCMA; and the second binding protein binds to CD38, CD138, or CS-1.

15. An engineered cell according to claims 12, wherein the first binding protein binds to CD123; and the second binding protein binds to CD33 or CLL1.

16. An engineered cell according to claims 12, wherein the first binding protein binds to PSCA; and the second binding protein binds to PSMA.

17. An engineered cell according to claims 12, wherein the engineered cell is a T-cell or NK cell.

18. The engineered cell of claim 12 comprising inactivated gene of PD-1, TIM3, or LAG3 by gene knockout method.

19. The engineered cell of claim 17, wherein the T-cell is a CD4 T-cell or CD8 T-cell.

20. The engineered cell according to claim 19, wherein the NK cell is an NKT cell or NK-92 cell.

21. A pharmaceutical composition, comprising the cell of any of claims 12-20 and a pharmaceutical acceptable carrier.

22. A method for treating cancer comprising administering to a subject in need thereof, a therapeutically effective amount of the cell of claim 12.

23. The method of claim 22, wherein the cancer is blood cancer.

24. The method of claim 22, wherein the cancer is lymphoma.

25. A method for stimulating a T cell-mediated immune response to a target cell population or tissue in a human, the method comprising administering to the human an effective amount of an engineered cell genetically modified to express a first polypeptide and a second polypeptide wherein the first polypeptide comprises a CD19 antigen binding domain, a transmembrane domain, a costimulatory signaling region of 4-1BB or ICOS, and a CD3 Zeta signaling domain, and wherein the second polypeptide comprises an immuno-regulatory cytokines or extracellular domain of cytokine receptors, a transmembrane domain.

26. A method of claim 25, wherein the CD19 antigen binding domain specifically binds to cancer cells expressing CD19.

27. A method of claim 25, wherein said second polypeptide comprises an IL7, a transmembrane domain.

28. A polynucleotide sequence encoding a chimeric antigen receptor (CAR), or a CAR system comprising a sequence selected from a group consisting of SEQ ID NO:6, 29, 31, 35, 37, 39, 41, 43, 45, 47, 49, 51, 53 and 55.

29. A CAR or CAR system comprising a polypeptide with a sequence selected from a group consisting of SEQ ID NO:20, 30, 32, 36, 38, 40, 42, 44, 46, 48, 50, 52, 54 and 56.

30. An engineered cell expressing the polypeptide of SEQ ID NO: 30 or SEQ ID NO:32.

31. A method for treating cancer comprising administering to a subject in need thereof, a therapeutically effective amount of the cell of claim 30, wherein said cancer is B cell lymphomas (NHL), acute lymphoblastic leukemia (ALL) or chronic lymphocytic leukemia (CLL).

32. An engineered cell expressing the polypeptide of SEQ ID NO:38 or SEQ ID NO:44.

33. A method for treating cancer comprising administering to a subject in need thereof, a therapeutically effective amount of the cell of claim 32, wherein said cancer is B cell lymphomas (NHL) or chronic lymphocytic leukemia (CLL).

34. An engineered cell expressing a polypeptide with a sequence selected from a group consisting of SEQ ID NO:40, SEQ ID NO:42, SEQ ID NO:46, and SEQ ID NO:48.

35. A method for treating cancer comprising administering to a subject in need thereof, a therapeutically effective amount of the cell of claim 34, wherein said cancer is B cell lymphomas (NHL), acute lymphoblastic leukemia (ALL) or chronic lymphocytic leukemia (CLL).

36. An engineered cell expressing the polypeptide of SEQ ID NO:54 or SEQ ID NO:56.

37. A method for treating cancer comprising administering to a subject in need thereof, a therapeutically effective amount of the cell of claim 36, wherein said cancer is Hodgkin Lymphoma, Systemic anaplastic large cell lymphoma, Primary cutaneous anaplastic large cell lymphoma (pcALCL) or CD30-expressing mycosis fungoides (MF).

38. The polynucleotide of claim 1 comprising a sequence selected from a group consisting of SEQ ID NO:6, 29, 31, 35, 37, 39, 41, 43, 45, 47, 49, 51, 53 and 55.

39. A polypeptide encoded by the polynucleotide of claim 1, comprising a sequence selected from a group consisting of SEQ ID NO:20, 30, 32, 36, 38, 40, 42, 44, 46, 48, 50, 52, 54 and 56.
